(19) United States
(12) Patent Application Publication
(10) Pub. No.: US 2002/0068325 A1
(43) Pub. Date: Jun. 6, 2002

(54) Methods and compositions for highly efficient production of heterologous proteins in yeast

(76) Inventors: Davis T.W. Ng, State College, PA (US); Shilpa Vashist, State College, PA (US)

Correspondence Address: MCKEE, VOORHEES & SEASE, P.L.C., ATTN: PENNSYLVANIA STATE UNIVERSITY, 801 GRAND AVENUE, SUITE 3200, DES MOINES, IA, 50309-2721, US

(21) Appl. No.: 10/004,968

(22) Filed: Dec. 5, 2001

Related U.S. Application Data
(60) Provisional application No. 60/251,374, filed on Dec. 5, 2000.

(51) Int. Cl. C12P 21/02; C12N 1/18; C12N 15/74

(52) U.S. Cl. 435/69.1; 435/483; 435/254.2

(57) ABSTRACT

The invention provides methods and compositions for the highly efficient production of heterologous proteins in yeast and other fungi by overcoming the previous problems associated with failure of these proteins to fold properly. According to the invention, the quality control mechanism employed by fungi which returns misfolded proteins to the cytosol for degradation is manipulated so that these proteins are instead secreted.

### CROSS-REFERENCE TO RELATED APPLICATIONS

This application claims benefit under 35 U.S.C. § 119(e) of provisional application 60/251,374 filed Dec. 5, 2000.

### GRANT REFERENCE

Work for this invention was funded in part by Grant No. 428-12 80AA NIHA Proteins. The Government may have certain rights in this invention.

### FIELD OF THE INVENTION

This invention relates generally to the field of molecular biology. More specifically, this invention relates to the characterization of novel methods for the highly effective production of heterologous proteins in yeast and other fungi by manipulating protein processing by the endoplasmic reticulum. The methods of the invention can be used for large scale production of heterologous proteins and includes methods and as well as novel vectors for the same.

### BACKGROUND OF THE INVENTION

The development of gene manipulation technology has made it possible to produce useful proteins in large amounts with microorganisms. Prokaryotes such as Escherichia coli or Bacillus subtilis have been widely used as hosts due to their well established genetics. Most biological molecules of pharmaceutical interest, however, are proteins secreted from eukaryotic cells, which are often are not functional when produced by prokaryotic cells. The production of desirable eucaryotic proteins such as hormones, antibodies, clotting factors, proteases, enzymes, growth factors and inhibitors as well as molecules of pathogens used for vaccination at industrial scale has thus been problematic. Ideally, organisms that can be grown inexpensively by fermentation can be used to produce these molecules yet expression systems such as bacteria lack the secretory apparatus employed by eukaryotes and are thus unable to properly synthesize these types of proteins.

Yeasts, as single cell eukaryotes, seemed quite promising for this problem, as yeast has a normal secretory pathway common to all eukaryotes. This approach has met with only limited success since most heterologous proteins are either mislocalized or fail to properly fold. One strategy to help in proper localization is by fusing an endogenous signal sequence to direct transport of the heterologous protein into the endoplasmic reticulum, the first step of the secretory pathway. This helped with the localization problem but it was found that most heterologous proteins properly transported into this compartment even with the aid of an endogenous signal sequence still fail to fold. Under these circumstances, synthesis using mammalian tissue culture has been the only practical choice. Unfortunately, the growth media and equipment required makes this a highly expensive and complex option.

Yeasts also represent high safety, since Saccharomyces has been long used for the production of fermentation products such as alcoholic products or bread. Yeast can generally be cultured at a cell density higher than bacteria as well as in a continuous mode. Yeast also provides for glycosylation of secreted proteins when exported into the medium thus preserving activity for proteins which require this modification for activity. However, it remains why so many secretory proteins from other organisms fail to produce active proteins when made in yeast and it has remained an unreliable expression system for these types of proteins.

As can be seen from the foregoing, there is a continuing need in the art for development of effective, convenient, and expeditious transformation systems which allow one to take advantage of the benefits of production in yeast without the protein misfolding problems.

It is thus an object of the present invention to provide transformation strategies for yeast that will accomplish the foregoing need.

A further object of this invention is to provide mechanisms for application of transgenic techniques such as those applied to bacteria, to produce heterologous proteins commercially.

It is yet another object of the invention to provide polynucleotide constructs, vectors, transformed cells for use in such transgenic protocols.

Other objects of the invention will become apparent from the description of the invention that follows.

### SUMMARY OF THE INVENTION

The method enables the genetic modification of yeast to facilitate their use as serve as biofermentors for the mass-scale production of commercially-important protein products, as for one example, human growth hormone. The invention promotes the proper synthesis of heterologous secretory proteins in yeast by overcoming the previous problems associated with the yeast expression system where many heterologous proteins fail to fold. In addition, this invention improves the yields and activity of proteins where yeast expression had shown some success. In short, this invention allows the production of heterologous proteins in yeast to be more similar (if not identical) to the proteins synthesized in the original host organism.

With the transformation methods of the invention, genetic engineering techniques known in the art and routinely applied to bacteria, plants, and animals can be used to genetically manipulate yeast production of recombinant proteins for harvest.

According to the invention, the quality control mechanism employed by yeast which returns misfolded proteins to the cytosol for degradation is manipulated so that these proteins are instead secreted. In a preferred embodiment the invention comprises the use of recipient yeast cell which has been manipulated so that an enzyme associated with O-glycosylation or the Bypass of Sec Thirteen families are inhibited. As a part of quality control, proteins with yeast specific modifications are eliminated. Inhibition of O-glycosylation prevents improper yeast specific modification thereby avoiding the yeast quality control mechanisms. Any method may be used according to the invention to generate the recipient host cells of the invention including deletion mutants, antisense or even administration of exogenous agonists or antagonists of enzymes involved in the regulatory pathways of these enzyme families.

The invention further comprises novel compositions including protein products isolated from such transgenic yeast. Also included are expression constructs, for use in this procedure as well as transformed cells, vectors, and transgenic yeast cells incorporating the same. In a preferred embodiment a new vector has been designed which helps to facilitate production of transgenic proteins in yeast.

Definitions

Various terms relating to the compositions and methods of the present invention are used herein above and also throughout the specification and claims and unless otherwise indicated shall have the meaning specified herein.

Various units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

An “antisense oligonucleotide” is a molecule of at least 6 contiguous nucleotides, preferably complementary to DNA (antigene) or RNA (antisense), which interferes with the process of transcription or translation of endogenous proteins so that gene products are inhibited.

A “cloning vector” is a DNA molecule such as a plasmid, cosmid, or bacterial phage that has the capability of replicating autonomously in a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include those that provide resistance to antibiotics such as hygromycin, tetracycline, or ampicillin.

A “coding sequence” or “coding region” refers to a nucleic acid molecule having sequence information necessary to produce a gene product, when the sequence is expressed.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid that encodes a polypeptide of the present invention is implicit in each described polypeptide sequence and is within the scope of the present invention.

As to amino acid sequences, one of skill will recognize that individual substitutions, deletions, or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W). See also, Creighton (1984) Proteins W. H. Freeman and Company.

The term “co-suppression” is a method of inhibiting gene expression in organisms wherein a construct is introduced to an organism. The construct has one or more copies of sequence that is identical to or that shares nucleotide homology with a resident gene.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both plant and fungi species, sequences can be modified to account for the specific codon preferences and GC content preferences as these preferences have been shown to differ, as described in the references cited herein.

The term “expression” refers to biosynthesis of a gene product. Structural gene expression involves transcription of the structural gene into mRNA and then translation of the mRNA into one or more polypeptides.

An “expression vector” is a DNA molecule comprising a gene that is expressed in a host cell. Typically, gene expression is placed under the control of certain regulatory elements including promoters, tissue specific regulatory elements, and enhancers. Such a gene is said to be “operably linked to” the regulatory elements.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

As used herein the term “high stringency” shall mean conditions or hybridization equivalent to the following: hybridized for 12 hours at 42° C. in a buffer containing 50% formamide, 5× SSPE, 2% SDS, 10× Denhardt's solution, and 100 μg/ml salmon sperm DNA, and washing with 0.1× SSC, 0.1% SDS at 55° C. and exposed to Kodak X-Omat AR film for 4 days at −70° C.

By “host cell” is meant a cell that contains a vector and supports the replication and/or expression of the vector. Host cells may be prokaryotic cells such as E. coli, or eukaryotic cells such as fungi, insect, amphibian, or mammalian cells. Preferably, the host cells are fungal cells.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “polynucleotide construct” or “DNA construct” is sometimes used to refer to an expression construction. This also includes, however, antisense oligonucleotides or nucleotides designed for co-suppression of native host cell sequences or extrinsic sequences corresponding, for example, to those found in viruses.

The term “operably linked” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, phosphorylation, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslation events, including natural processing event and events brought about by human manipulation, which do not occur naturally. Circular, branched, and branched circular polypeptides may be synthesized by a non-translation natural process and by entirely synthetic methods, as well. Further, this invention contemplates the use of both the methionine-containing and the methionine-less amino terminal variants of the protein of the invention. With respect to a protein, the term “N-terminal region” shall include approximately 50 amino acids adjacent to the amino terminal end of a protein.

The terms “promoter”, “promoter region”, or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase. The term promoter includes the essential regulatory features of said sequence and may optionally include a long terminal repeat region prior to the translation start site.

A “recombinant host” may be any prokaryotic or eukaryotic cell that contains either a cloning vector or an expression vector. This term also includes those prokaryotic or eukaryotic cells that have been genetically engineered to contain the clone genes in the chromosome or genome of the host cell.

The term “reporter gene” refers to a gene that encodes a product that is easily detectable by standard methods, either directly or indirectly.

The term “selectable marker gene” refers to a gene encoding a product that, when expressed, confers a selectable phenotype such as antibiotic resistance on a transformed cell.

With respect to oligonucleotides or other single-stranded nucleic acid molecules, the term “specifically hybridizing” refers to the association between two single-stranded nucleic acid molecules of sufficiently complementary sequence to permit such hybridization under pre-determined conditions generally used in the art i.e., conditions of stringency (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

A “structural gene” is a DNA sequence that is transcribed into messenger RNA (mRNA), which is then translated into a sequence of amino acids characteristic of a specific polypeptide.

A “vector” is a replicon, such as plasmid, phage, cosmid, or virus to which another nucleic acid segment may be operably inserted so as to bring about the replication or expression of the segment.

### DESCRIPTION OF FIGURES

FIG. 1 depicts the expression of KHN in yeast. KHN was expressed wild-type cells and the ER-associated degradation mutant cue1. Cells were pulse-labeled with ³⁵S amino acids and chased for the times shown. KHN was then immunoprecipitated from detergent lysates and resolved by SDS-PAGE followed by visualization by autoradiography.

FIG. 2 depicts the removal of N-linked sugars from KHN using endoglycosidase H. KHN expressed in cue1 cells were pulse-labeled with 35S-amino acids and chased for the times shown. KHN was immunoprecipitated and treated or mock treated with endo H. KHN was then resolved by SDS-PAGE and visualized by autoradiography.

FIG. 3 depicts KHN is modified by O-linked glycosylation. KHN is expressed in cue1, pmt2, and pmt1 mutant strains. Cells were pulse-labeled and chased as described. KHN was immunoprecipitated and analyzed as described in FIG. 1.

FIG. 4 is a graph depicting cells mutant for the BST1 gene as well as the PMT2 gene show dramatic improvement in KGFP activity as compared with wild type. The mean fluorescence intensity is 5-fold in the Δbst1 cells and 9-fold in the Δpmt2 cells.

FIG. 5. Fluorescence microscopy of KGFP-expressing cells. Wild-type and pmt2 mutant cells expressing KGFP were photographed using a Zeiss Axioplan epifluorescence microscope coupled with a Spot II digital camera. Exposure times are as shown.

FIG. 6. KHN is a rapidly degraded protein that is transported to the Golgi apparatus. (A) Wild-type and Acuel cells expressing KHN were metabolically pulse-labeled at 30 with [³⁵S] methionine/cysteine for 10 min followed by a cold chase for times indicated. KHN was immunoprecipitated from detergent lysates using anti-HN polyclonal antiserum and resolved by electrophoresis on a 10% SDS polyacrylamide gel. Where indicated, N-linked carbohydrates were removed by incubation in immunoprecipitated proteins with 500 U endoglycosidase H (Endo H) for 3 h. The positions of proteins immunoprecipitated nonspecifically are indicated by asterisks. (B) Wild-type Δpmt1, and Δpmt2 cells expressing KHN were analyzed as described for A. (C) Wild-type, sec12-4, and sec18-1 cells expressing KHN were grown to log phase at 22° C. and shifted to 37° C. After 30 min, the cells were pulse-labeled and chased for the times indicated. KHN was immunoprecipitated and analyzed as described for A. The positions of the KHN p1 and p2 forms are indicated (A), and arrows mark the position of the p1 form (B and C).

FIG. 7. KHNₜ is a substrate for degradation by the ERAD pathway. (A) Wild-type and mutant strains expressing KHNₜ were pulse-labeled for 10 min with [³⁵S] methionine/cysteine and followed by a cold chase as indicated. Immunoprecipitation of KHNₜ was performed using anti-HA monoclonal antibody (HA.11; BabCo) and normalized by total TCA precipitable counts. Proteins were analyzed by SDS-PAGE and visualized by autoradiography. (B) The experiments described for A were quantified by Phosphorlmager analysis using the same gels that generated the autoradiograms shown in A. (C) Relative steady-state levels of KHNₜ in wild-type and ERAD mutants were analyzed by immunoblotting. Equal amounts of cell lysate (0.2 OD₆₀₀ equivalents of cells) were loaded in each lane, separated by electrophoresis, transferred to nitrocellulose, and probed using HA.11 monoclonal antibody. Proteins were visualized using chemiluminescence (Pierce Chemical Co.). (D) Immunolocalization of KHNₜ in wild-type and ERAD mutant cells were performed using fixed and permeabilized cells on glass slides. KHNₜ and BiP were detected using α-HA monoclonal antibody and α-Kar2p polyclonal antiserum, respectively. After binding of fluorescent secondary antibodies, KHNₜ was visualized in the red channel (“a, b, and c), and BiP was visualized in the green channel (d, e, and f). In each channel, images were captured using identical exposure times. Bar, 2 μm.

FIG. 8. ER-to-Golgi transport is required for degradation of soluble but not membrane-bound ERAD substrates. (A-D)) Wild-type and ER transport mutant strains sec12-4 and sec18-1 expression HA-tagged ERAD substrates were grown to log phase at 22° C. and shifted to the restrictive temperature of 37° C. for 30 min. Time courses were performed and analyzed as described in the legend to FIG. 7. The data is plotted to compare rates of degradation for each substrate in various strain backgrounds. A Δcue1 strain was included as a positive control for Ste6-166p and Sec61-2p.

FIG. 9. Soluble ERAD substrates are contained in COPII vesicles. Reconstituted COPII budding reactions were performed on ER membranes isolated from wild-type strains expressing KHNₜ (A), CPY*_{HA} (B), and Ste6-166p (C). Lanes labeled T represent one tenth of the total membranes used in a budding reaction, minus (−) lanes indicate the amount of vesicles formed in the absence of the purified COPII components, and plus (+) lanes indicate vesicles produced when COPII proteins are added. Total membranes and budded vesicles were collected by centrifugation, resolved on a polyacrylamide gel, and immunoblotted for indicated proteins. The amount of glyco-pro-α-factor (gpαf) was detected using fluorography.

FIG. 10. Degradation of KHNₜ and CPY*_{HA} but not Ste6-166p requires Golgi-to-ER transport. Pulse-chase analysis was performed on wild-type and sec21-l strains expression (A) KHNₜ, (B) CPY*_{HA}, and (C) Ste6-166p as described in the legend to FIG. 2 except that strains were grown to log phase at 22° C. and pulse-labeled immediately after a shift to 33° C. Incubation at 33° C. was continued for the cold chase (times as indicated). Gels were visualized by autoradiography (left) and quantified by Phosphorlmager analysis (right). In C, the gel images were from Phosphorlmager scans.

FIG. 11. per17-1 is a mutant specific to the retrieval pathway, which blocks the transport of misfolded proteins but not properly folded proteins. (A) The turnover of KHNₜ, CPY*_{HA}, Ste6-166p, and Sec61-2p in wild-type and per17-1 cells were measured by metabolic pulse-chase analysis as described in the legend to FIG. 7. Experiments were performed at 30° C. except for strains expressing Sec61-2p. Strains expressing Sec61-2p were grown to log phase at 30° C., shifted to 37° C. for 30 min, and continued for the pulse-chase. (B) Autoradiograms generated from gels of the KHNₜ time course shown in part A are shown at the top. The positions of the p1 (ER) and p2 (Golgi-modified) forms are indicated. Endogenous CPY and Gas1p were immuno-precipitated in parallel from aliquots of lysates prepared from the KHNₜ time course. The proteins were separated by gel electrophoresis and visualized by autoradiography (P1, ER proCPY; P2, Golgi proCPY; mCPY, mature CPY; ER Gas 1p, ER form of Gas1p; mGas1, mature Golgi-modified Gas1p). (C) Wild-type and per17-1 cells were pulse labeled for 10 min and chased for times indicated. CPS and ALP were immuno-precipitated and analyzed by gel electrophoresis followed by autoradiography. The pro (proCPS and proALP) and mature (mCPS and mALP) forms of each protein are indicated.

FIG. 12. Immunolocalization of misfolded proteins in per17-1 cells. (A) per17-1 cells expressing KHNₜ (a-c) and CPY*_{HA} (d-f) and Δder1 cells expressing CPY*_{HA} (g-I) were fixed and permeabilized from logarithmic cultures. The cells were stained with α-HA and α-Kar2p antibodies followed by Alexa Fluor 546 goat α-mouse (a, d, and g) and Alexa Fluor 488 goat α-rabbit (b, e, and h) secondary antibodies. Staining with DAPI (c, f, and I) indicates the positions of nuclei. Arrows mark specific points of colocalization. (B) Wild-type and per17-1 cells expressing HA epitope-tagged SRβ were processed and bound to primary antibodies as in A. Alexa Fluor 546 goat α-rabbit and Alexa Fluor 488 goat α-mouse were used such that BiP was visualized in the red channel (a and d), whereas SRβ was visualized in the green channel (b and e). Bars, 2 μM.

FIG. 13. Proposed model of ER quality control in budding yeast. After translocation, proteins that misfold are sorted for the retention pathway (white arrows) or the retrieval pathway (black arrows). In the retrieval pathway, proteins are packaged into COPII vesicles, transported to the Golgi apparatus, and retrieved via the retrograde transport pathway. In the ER, substrates of both pathways converge for ERAD. The proteins cross the ER membrane via the translocon complex, marked by ubiquitination and degraded by the cytosolic 26S proteasome.

FIG. 14 is a plasmid map of pDN477, a yeast expression vector that allows the high level expression of heterologous proteins in yeast. Messenger RNA synthesis is driven by the powerful TDH3 promoter (shown). Included is the signal sequence (‘SS’) from the yeast BiP (KAR2) gene that directs the translocation of protein into the cotranslational (and more mammalian) SRP secretion pathway by inserting the cDNA into the Clal (5′) and Xbal (3′) sites. To avoid secretion or to use an endogenous signal sequence, insert coding sequences into the BamH1 (5′) and Xbal (3′) sites. Transcription is terminated by the ACT1 terminator. The vector also contains the URA3 gene for selection in yeast and yeast origin of replication (ARS1) and centromere (CEN4). Versions of pDN477 with other markers or for integration into the genome are available.

### DETAILED DESCRIPTION OF THE INVENTION

This invention was developed from studies to understand the process of secretory protein folding and maturation in the yeast. In the course of the studies, a number of heterologous proteins were expressed in the yeast secretory pathway. The first was the green fluorescent protein (GFP) from jellyfish. To direct it into the secretory pathway, an endogenous yeast signal sequence from the Kar2p protein was fused to the amino-terminus of GFP. This signal sequence will direct a protein into a specific translocation pathway, Kar2p utilizes the more “mammalian” SRP pathway in yeast. This signal sequence is preferred as opposed to the commonly used alpha-factor signal sequence which uses the yeast-specific posttranslational pathway. In addition, the endoplasmic reticulum (ER) retention motif HDEL was fused to the carboxyl-terminus to localize the protein to the ER. GFP is an ideal molecule to monitor protein folding since its fluorescence activity is dependent on correct protein conformation and can be easily measured. When expressed, the chimeric protein called KGFP is properly localized but the fluorescence activity is very low suggesting it is not folding properly in the ER. This low activity is specific to expression in the secretory pathway since expression in the cytosol using the ER translocation mutant sec63 shows brilliant cytosolic fluorescence. It was unclear why KGFP fails to fold efficiently in the yeast secretory pathway.

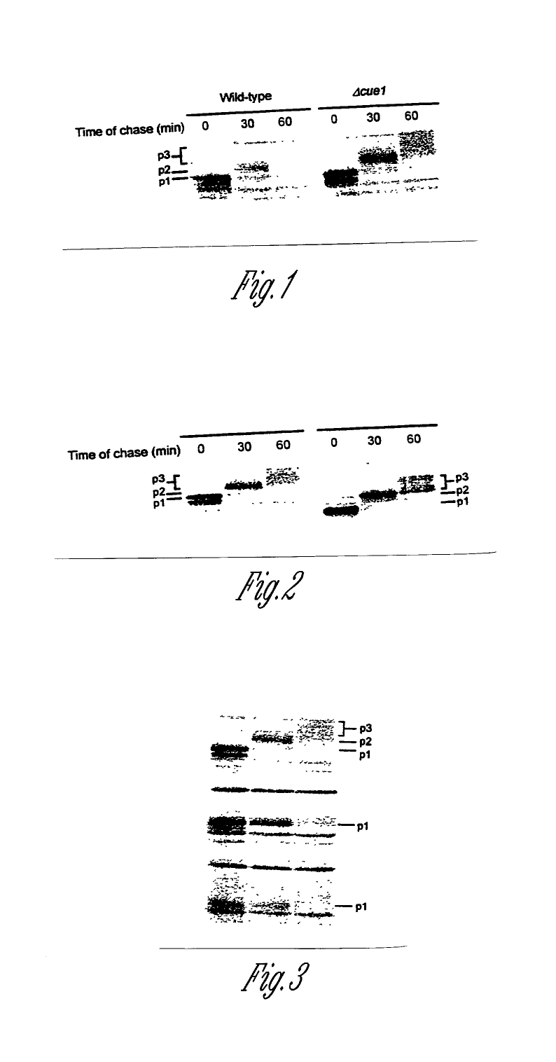

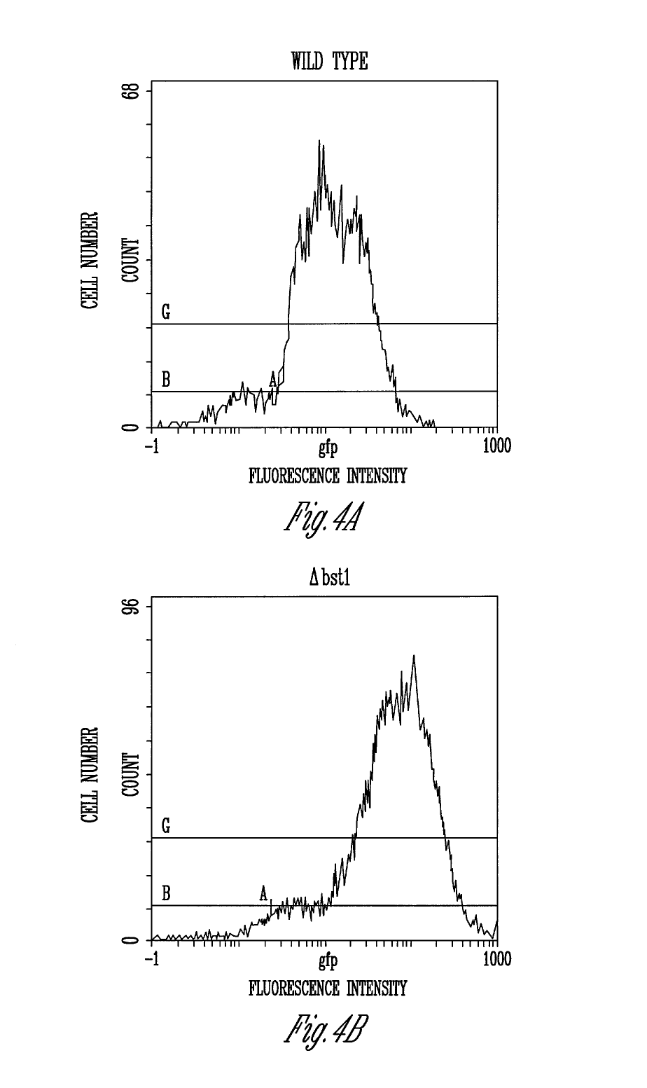

The breakthrough came about when a mammalian virus glycoprotein from simian virus 5 called HN was expressed. HN was chosen since its folding can be easily monitored. To express HN in yeast, the viral signal/anchor domain (it was not recognized in yeast) was replaced with the Kar2p signal sequence. The resulting protein called KHN is properly targeted to the secretory pathway as it was efficiently glycosylated (FIG. 1).

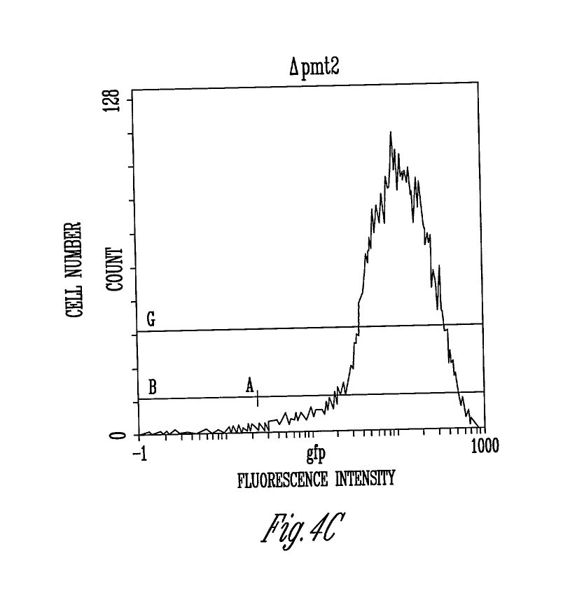

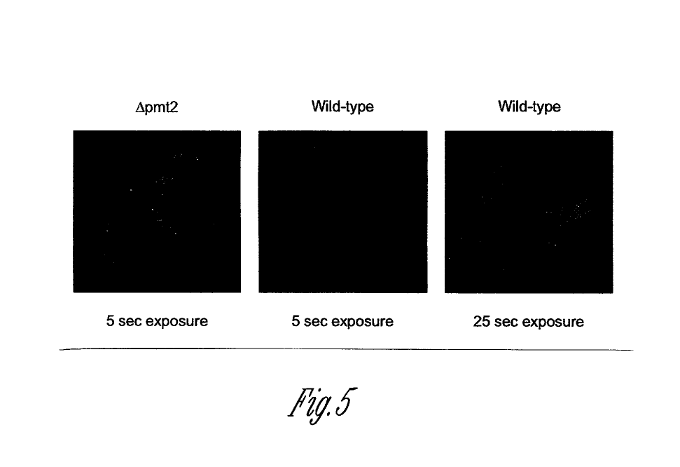

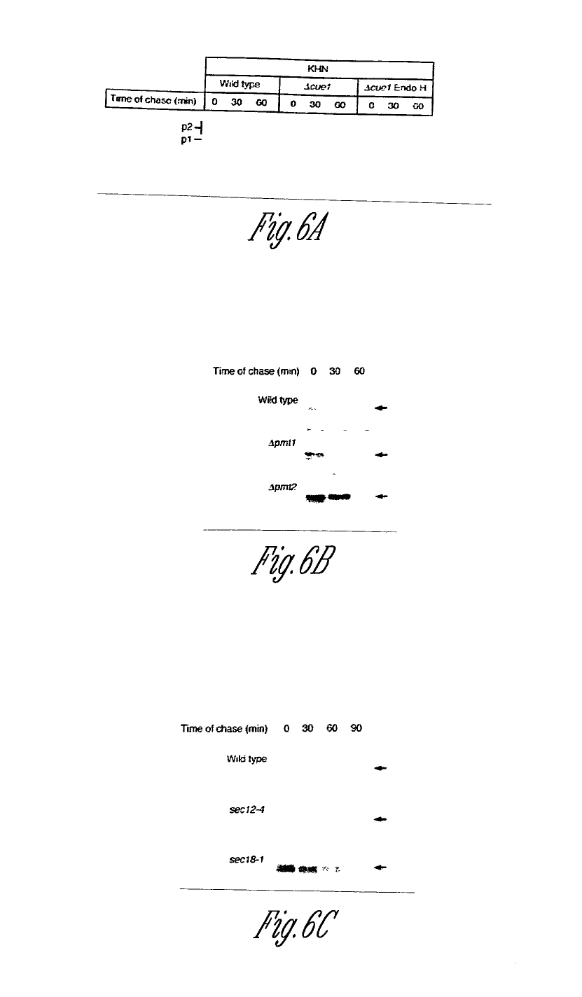

The protein was rapidly degraded (FIG. 1). This occurs commonly to proteins that are misfolded in the ER. This was confirmed when we found KHN to be stabilized by the ubiquitination mutant cue1 that is defective for ER-associated protein degradation (FIG. 1). A time-dependent shift in mobility was also observed that is indicative of modification of carbohydrates (KHN is a glycoprotein). Thus it was tested whether the shift was due to modification of N-linked carbohydrates by digesting KHN with endoglycosidase H. As shown in FIG. 2, the shift is not due to the modification of KHN N-linked sugars. The other possibility is that KHN is modified by O-linked sugars. This surprising since HN is normally only modified by N-linked sugars in its normal mammalian host. This possiblility was tested by expressing KHN in yeast strains defective for O-linked glycosylation. In yeast, O-linked glycosylation begins in the ER through the action of a family of genes called protein mannosyltransferases (PMT). Surprisingly, the inventors found the modification in HN to be blocked in two of these mutants pmt1 and pmt2 showing that KHN is inappropriately modified by O-linked glycosylation (FIG. 3).

In higher eukaryotes, O-linked glycosylation is a rare modification that occurs in the Golgi apparatus. Thus, all polypeptides are folded prior to any addition of O-linked sugars. By contrast, the first step of O-linked glycosylation occurs in the ER of yeast cells. However, it is not known what signals O-linked glycosylation and it is possible that most heterologous proteins can become O-linked glycosylated. As it was not previously known, the inventors hypothesized that the inappropriate modification of nascent polypeptides in the ER by O-linked glycosylation may change the chemical nature of the chain and potentially cause misfolding. At best, even if the protein can fold with the modification, the activity or stability of the protein may be compromised since it is chemically different from the native form. To test this hypothesis, we tested the effect of inhibiting O-linked glycosylation on folding using our reporter construct KGFP (KHN is less ideal for this purpose since it is a soluble version of the native HN and is partially compromised for folding in mammalian cells).

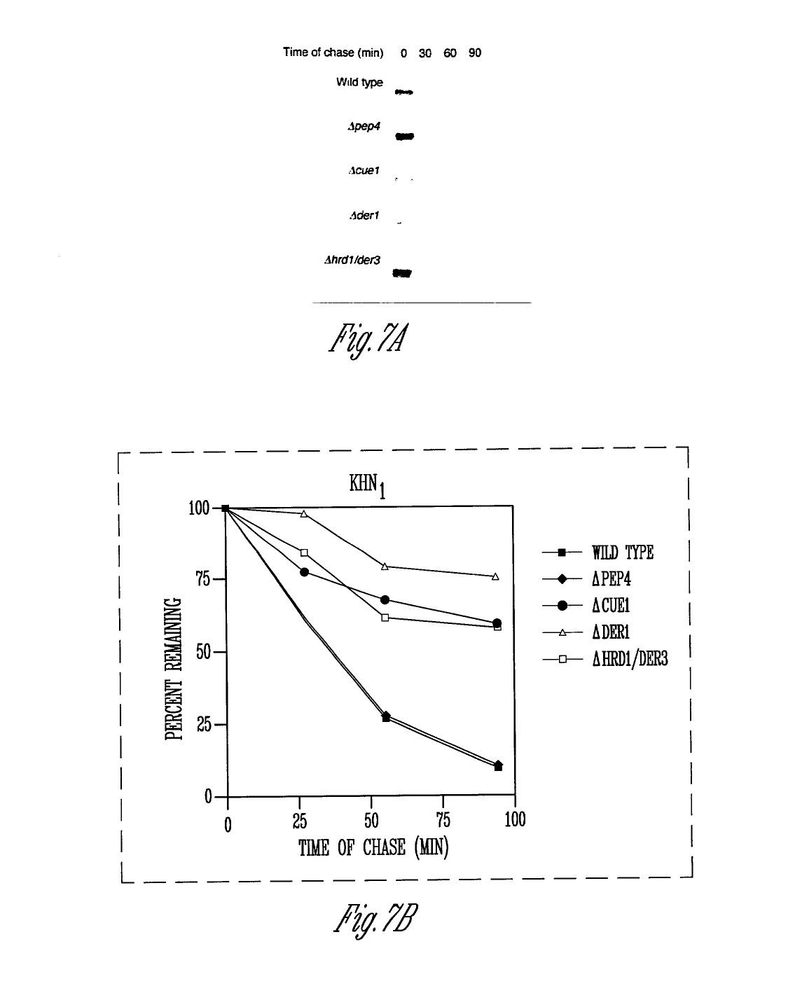

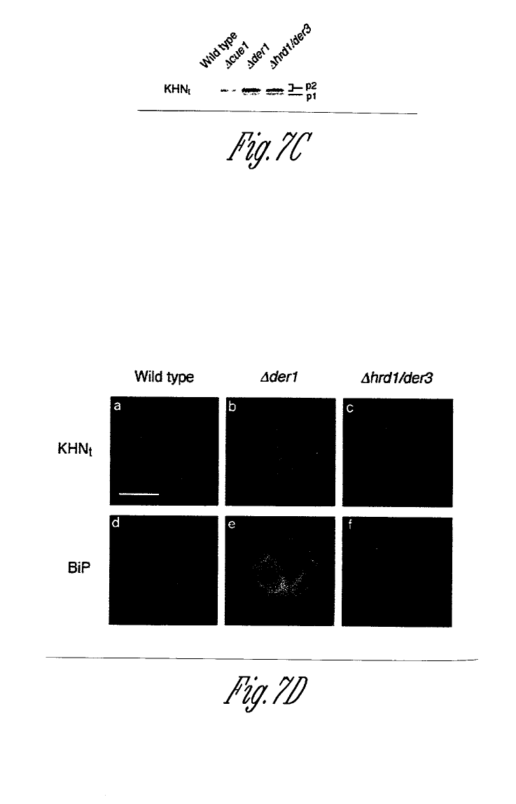

KGFP was expressed driven by the yeast TDH3 promoter in wild type and pmt mutant cells. Since KGFP is a fluorescent marker, folding could be monitored by changes in emission intensity. KGFP was visually screened in expressing cells using an epifluorescence microscope. In all cases, KHN was properly targeted to the ER. Interestingly, the pmt2 mutant had the strongest effect. It exhibited a much brighter ER staining pattern than control. Other pmt mutants 4 and 3 showed a lesser effect. To quantify and characterize the apparent increase in fluorescence activity, flow cytometry was performed on wild type and pmt2 mutant cells expressing KGFP. As shown in FIG. 4, fluorescence activity in pmt2 cells showed uniform increase over wild-type cells. The average activity is nearly 8.5-fold higher in the pmt2 mutant (109.5 units vs. 12.9 units) and for bst1, there is a 5.5 fold increase (71.4 units vs. 12.9). This difference can be attributed to a difference in specific activity since quantitative pulse-chase analysis shows that expression levels and stability is similar in both strains. In addition, direct fluorescence microscopy shows the dramatic improvement in activity and that the improvement is not due to mislocalization of KGFP (FIG. 5).

These data show that heterologous proteins expressed in yeast are inappropriately modified by O-linked glycosylation. In turn, the modification can have negative consequences on the maturation and activity of the protein. The inventors have established that coupling expression using an endogenous signal sequence with specific mutant strains deficient in O-linked glycosylation, the activity of heterologous proteins expressed in yeast can be drastically improved. Since there are 6 PMT genes in yeast that are non-redundant and exhibit differences in substrate specificity, deletion strains of any of the six genes may provide the needed inhibition of aberrant O-glycosylation. In addition, mutations can be combined to further promote proper folding. Thus the inventors have developed a novel solution for overcoming a problem that has limited the potential of low cost expression of commercially important molecules in yeast.

Currently, a variety of expression systems exist for the synthesis of proteins at a preparative scale. The most common organism E. coli, is generally not useful for the synthesis of eukaryotic secretory proteins as bacterial secretion is fundamentally different. The yeast system has been of limited use since many proteins are not faithfully synthesized for previously unknown reasons. According to the invention an observation that a soluble form of the viral glycoprotein SV5 HN is inappropriately modified in yeast cells resulting in its misfolding has been exploited to overcome this problem. The inventors show that inhibiting O-linked glycosylation, (the examples show specific mutant strains but any method of inhibition is expected to have the same effect), the synthesis of active heterologous proteins can be dramatically enhanced. In addition, the use of an endogenous cotranslational-specific signal sequence that is more “mammalian-like” may also preferably be used to direct the correct targeting to the yeast ER. Although this approach was developed in S. cerevisiae, it is applicable to all other yeast including S. pombe and P. pastoris as well as other fungi since they all share the machinery to O-glycosylate proteins in the ER.

This system has a wide application of use since virtually any heterologous protein (secretory or not) can be synthesized including but not limited to antibodies, hormones, growth factors and inhibitors, toxins, clotting factors, enzymes, and proteins for immunization. In addition to the applications for large-scale protein synthesis, the invention will allow yeast to be used as a powerful research tool for study and drug screens using proteins implicated in human disease. These include but are not limited to the cystic fibrosis transmembrane conductance regulator (CFTR), prion proteins, the expression of cellular receptors to screen for agonists and antagonists, and the processing of the β-amyloid precursor protein of Alzheimer's disease.

According to the invention yeast transformation is conducted in an environment where the quality control mechanisms are inhibited or manipulated so that proteins are not degraded by traditional pathways in the Golgi and ER. In a preferred embodiment the recipient cell environment is one in which O-glycosylation is inhibited. This can be accomplished through the use of antisense or cosuppression as known in the art, or through the engineering of yeast host strains that have loss of function mutations in genes associated with O-linked glycosylation. In a preferred embodiment O-linked glycosylation is inhibited via manipulation of the PMT family of genes.

In another embodiment the quality control mechanisms are manipulated by mutation or inhibition of the Bypass of Sec Thirteen gene or other similarly functioning genes.

Antisense and cosuppression mechanisms are commonly known and used in the art and described for example in Ausubel et al supra. In addition, techniques for constructing mutations in recipient yeast cell lines are also known and standard in the art as described in Sambrook et al 1989. These include such techniques as integrative disruption Shortle, 1982 Science 217:373 “Lethal Disruption of the Yeast Actin Gene of Integrative DNA Transformation”; one step gene disruption Rothstein 1983, Methods Enzymol. 101:202-210 “One Step gene Disruption in Yeast”; PCR Mediated One Step Gene Disruption Baudin et al, 1993, Saccharomyces cerevisiae. Nucl. Acids Res. 21:3329-3330, “A Simple and Efficient Method for Direct gene Deletion in Saccaromyces cerevisiae”; or Transplacement Scherer and Davis 1979, PNAS 76:4951-4955 “Replacement of Chromosome Segments with Altered DNA Sequences Constructed in vitro”. In preferred embodiment the recipient environment with manipulation of Er quality control is created by engineering a deletion mutant yeast or fungi recipient strain which is deficient in a gene necessary for proper quality control.

In a preferred embodiment the gene is the ByPass or Sec Thirteen gene, Elrod-Erickson and Kaiser (1996, Molecular biology of the Cell, 7:1043). It is expected that other such genes will be identified in yeast in the BST family that will serve similar function and will be useful according to the invention. One may identify other yeast BST genes by using known sequences from other species, generating probes and hybridizing with libraries according to teachings well know in the art and disclosed in herein and in Ausubel, Protocols in Molecular Biology 1997, Wiley and Sons.

In another preferred embodiment the recipient yeast cell has been manipulated so that o-mannosylation is inhibited. This can be accomplished by inhibiting any enzyme in the o-linked glycosylation pathway. Protein O-mannosylation, originally observed in fungi, starts at the endoplasmic reticulum with the transfer of mannose from dolichol activated mannose of seryl or threonyl residues of secretory proteins. This reaction is catalyzed by a family of protein O-mannosyltransferases (PMT) See, Protein O-mannosylation, Biochimica et Biophysica Acta 1426 (1999) 297-307, Strahl-Bolsinger et al.

In a preferred embodiment the enzyme which is inhibited is of the PMT family of genes. There are currently at least 6 or more known protein O-glycosylation genes PMT 1-7. See The PMT gene family: protein O-glycosylation in Saccharomyces cerevisiae is vital” The EMBO Journal Gentzsch et al, vol 15, no. 21 pp. 5752-5759 1996.

Protein o-mannosylation is the first step in O-linked glycosylation, inhibition of other steps in this pathway would be expected to give similar results according to the invention. See Hersgovics et al, “Glycoprotein Biosynthesis in Yeast” The FASEB Journal Vol. 7 1993 pgs 540-550. This may for example include inhibition of the MNT/KRE2 gene family (KTR1 and YUR1) which catalyze attachment of the third mannose residue. Other O-linked glycosylation mutants may be easily screened using the protocols herein to identify other mutants which will work according to the invention with no more than routine screening.

Production of recombinant proteins in yeast combines the teachings of the present disclosure with a variety of techniques and expedients known in the art. The invention further comprises the use of polynucleotides which encode structural genes the expression of which is desired in a host fungi cell. These polynucleotides are often in the form of an expression construct which incorporates promoter regions operably linked to the structural gene and often termination sequences. The construct may also include signal sequences to direct secretion of the transgenic protein. The construct is usually contained within a vector, usually a plasmid vector which may include features for replication and maintenance of the vector in bacteria (cloning vector) a selectable marker gene and/or sequences for integration and/or function in a host (expression vector).

Each of these components as used in the methods of the invention is intended to be within the scope of the invention. In most instances, alternate expedients exist for each stage of the overall process. The choice of expedients depends on the variables such as the plasmid vector system chosen for the cloning and introduction of the recombinant DNA molecule, the yeast species to be modified, the particular structural gene, promoter elements, and upstream elements used. Persons skilled in the art are able to select and use appropriate alternatives to achieve functionality. Culture conditions for expressing desired structural genes and cultured cells are known in the art. Also as known in the art, a number of yeast species are transformable and fermentable such that the cells, containing and expressing desired genes under regulatory control of the promoter molecules according to the invention may be obtained.

The following is a non-limiting general overview of molecular biology techniques that may be used in performing the methods of the invention.

The polynucleotide constructs of the present invention will share similar elements, which are well known in the art of molecular biology. For example, in each construct the DNA sequences of interest will preferably be operably linked (i.e., positioned to ensure the functioning of) to a promoter which is functional in a yeast cell and that allows the DNA to be transcribed (into an RNA transcript) and will comprise a vector that includes a replication system. In preferred embodiments, the DNA sequence of interest will be of exogenous origin in an effort to prevent co-suppression of the endogenous genes, unless co-suppression is the desired protocol.

Yeast Cloning Vectors and Genes

Based upon their mode of replication in yeast commonly used yeast vectors can be grouped into 5 categories. Yip, Yrp, Ycp, YTEp, and Ylp plasmids. With the exception of Ylp plasmids (yeast linear plasmids) all of these can be maintained in E. Coli. Plasmid Vector Development.

Three types of chimeric plasmid vectors were developed by Struhl et al. (Struhl, K., 1979, “High-frequency transformation of yeast: autonomous replication of hybrid DNA molecules”, Proc. Natl. Acad. Sci. USA 76:1035-1039): (i) YIp (yeast integrating plasmids), which are unable to replicate and transform by integration into the genome of the recipient strain; (ii) YEp (yeast episomal plasmids), which carry the replication origin of the yeast 2-μm circle, an endogenous yeast plasmid, and can replicate in the recipient cell; and (iii) YRp (yeast replicating plasmids), which can replicate utilizing yeast autonomous replicating sequences (ARS). Integrating vectors transformed with low efficiencies, 1-10 transformants/μg. Plasmids that could replicate in the yeast cell transformed with much higher efficiencies. The YEp vectors generally transform with an efficiency of 0.5-2.0×10⁴ transformants/μg input plasmid DNA, and the YRp7 plasmid produced 0.5-2.0×10³ transformants/μg input plasmid DNA. Struhl et al. (Struhl, K., 1979, “High-frequency transformation of yeast: autonomous replication of hybrid DNA molecules”, Proc. Natl. Acad. Sci. USA 76:1035-1039): (i) YIp (yeast integrating plasmids) demonstrated that plasmids that require integration into the genome transform less efficiently than those yeast plasmid vectors that can replicate autonomously in the yeast cell. Since then, two other yeast plasmid vectors have been developed. Yeast centromere plasmids (YCp) that carry an ARS and a yeast centromere (Clarke, L., et al., 1980, “Isolation of a yeast centromere and construction of functional small circular chromosomes”, Nature, 287:504-509; Parent, S. A., et al., 1985, “Vector systems for the expression, analysis and cloning of DNA sequences in S. cerevisiae”, Yeast J:83-138) are more stable than YRp plasmids but are present in only one copy per cell. Yeast artificial chromosomes (YACs) are propagated as a circular plasmid with a centromere and an ARS plus two selectable markers, two telomeres, and a cloning site (Burke, et al, 1987, “Cloning of large segments of exogenous DNA into yeast by means of artificial chromosome vectors”, Science 236:806-812; Murray, A. W., et al., 1983, “Construction of artificial chromosomes in yeast”, Nature, 305:189-193). The vector is linearized by the removal of a sequence between the telomeres, and foreign DNA is inserted into the cloning site. The result is a linear artificial chromosome, 100-1000 kb in length, that can be propagated through mitosis and meiosis.

Promoters

The expression constructs, promoters or control systems used in the methods of the invention may include an inducible promoter or a constitutive promoter. A large number of suitable promoter systems are available. Examples of inducible yeast promoters include GAL (galactokinase) and PHO5 (alkaline phophatase), Schneider and Guarente, 1991. The GAL promoter is activated by galactose while the PHO5 promoter is induced by a medium that lacks phosphate.

A constitutive promoter may also be employed. Examples of these include the ADH1 (alcohol dehydrogenase I) TPI (triose phosphate isomerase) and PGK (3phosphoglycerate kinase) are the most commonly used. See, Ausubel, Short Protocols in Molecular biology, 1999 John Wiley and Sons.

These and other such promoters are known and accessible through sources such as Genbank. In a preferred embodiment, the promoter is homologous to the recipient host cell species. For example, in a S. cerevisiae transformation protocol, an S. cerevisiae promoter may be used in the polynucleotide construct.

It may also be desirable to include some intron sequences in the promoter constructs since the inclusion of intron sequences in the coding region may result in enhanced expression and specificity.

Additionally, regions of one promoter may be joined to regions from a different promoter in order to obtain the desired promoter activity resulting in a chimeric promoter. Synthetic promoters that regulate gene expression may also be used.

The expression system may be further optimized by employing supplemental elements such as transcription terminators and/or enhancer elements.

Other Regulatory Elements

In addition to a promoter sequence, an expression cassette or polynucleotide construct should also contain a transcription termination region downstream of the structural gene to provide for efficient termination. The termination region or polyadenylation signal may be obtained from the same gene as the promoter sequence or may be obtained from different genes. Polyadenylation sequences include, but are not limited to the Agrobacterium octopine synthase signal (Gielen et al., EMBO J. (1984) 3:835-846) or the nopaline synthase signal (Depicker et al., Mol. and Appl. Genet. (1982) 1:561-573).

Transport of protein produced by transgenes to a subcellular compartment such as the vacuole, peroxisome, glyoxysome, cell wall or mitochondrion, or for secretion into the apoplast or growth medium, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately located. The presence of a signal sequence directs a polypeptide to either an intracellular organelle or subcellular compartment or for secretion to the apoplast or into the external environment. Many signal sequences are known in the art particularly for yeast such as BiP sequence. A sequence operably linked to a protein encoding sequence makes the resultant protein a secretory protein. The use of a signaling sequence for secretory proteins is preferred for the invention but the invention also is intended to cover traditionally processed proteins in addition to secretory proteins which are so directed by signal sequences.

Marker Genes

Recombinant DNA molecules containing any of the DNA sequences and promoters described herein may additionally contain selection marker genes that encode a selection gene product conferring on a cell resistance to a chemical agent or physiological stress, or confers a distinguishable phenotypic characteristic to the cells such that cells transformed with the recombinant DNA molecule may be easily selected using a selective agent. Selectable marker genes used in yeast transformation include URA3, LEU2, HIS3, and TRP1. These genes complement a particular metabolic defect (nutritional auxotrophy) in the yeast host. Markers that confer resistance to fungicides such as benomyl or eukaryotic poisons may also be used.

Replicators

The yeast expression vector may also include a replicator derived from the yeast 2 um circle which has DNA sites and genes which ensure proper copy number and proper segregation into daughter cells.

Proteins

With transgenic yeast according to the present invention, a foreign protein can be produced in commercial quantities. Thus, techniques for the selection and propagation of transformed yeast, which are well understood in the art, yield a plurality of transgenic yeast that are harvested in a conventional manner, and a foreign protein then can be extracted from a tissue of interest or from total biomass, or secreted into the growth medium (liquid or solid state) and then recovered. Protein extraction from plant and fungal biomass can be accomplished by known methods which are discussed, for example, by Heney and Orr, Anal. Biochem. 114: 92-6 (1981), and in the references cited herein.

Transformation

A number of standard protocols exist for yeast transformation and may be used according to the invention and are discussed below.

Spheroplast Transformation

Many methods exist for transformation yeast cells including the spheroplast method by which the yeast cell wall is removed, preferably enzymatically (by glusulase) before treatment with PEG and plasmid (preferably self replicating) DNA.

Sample Spheroplast Transformation Protocol

1. Cells are grown in 50 mL YPAD to a density of 3×10⁷ cells/mL.

2. The cells are harvested by centrifugation at 400×600× g for 5 min, washed twice in 20 mL sterile water, and washed once in 20 mL 1 M sorbitol. The cells are resuspended in 20 mL SPEM (1 M sorbitol, 10 mM sodium phosphate, pH 7.5, 10 mM EDTA plus 40 μl β-mercaptoethanol added immediately before use).

3. The cells are converted to spheroplasts by the addition of 45 μL zymolyase 20T (10 μg/mL) and incubation at 30° C. for 20-30 min with gentle shaking. By this time, 90% of the cells should be converted to spheroplasts.

4. The spheroplasts are harvested by centrifugation at 250× g for 4 min, and the supernatant is removed carefully. The pellet is washed once in 20 mL STC (1M sorbitol, 10 mM Tris-HC1, pH 7.5, 10 mM CaCl₂) and resuspended in 2 mL STC.

5. Spheroplasts are transformed by gently mixing 150 μl of the suspension in STC with 5 μg carrier DNA and up to 5 μg plasmid DNA in less than 10 μL. The mixture is incubated for 10 min at room temperature. One milliliter of PEG reagent [10 mM Tris-HC1, pH 7.5, 10 mM CaCl₂, 20% (w/v) PEG 8000; filter sterile] is added and mixed gently, and incubation is continued for another 10 min.

6. The spheroplasts are harvested by centrifugation for 4 min at 250× g and resuspended in 150 μL SOS (1.0 M sorbitol, 6.5 mM Cac1₂, 0.25% yeast extract, 0.5% bactopeptone). Dilution of spheroplasts are mixed with 8 mL TOP (selective medium containing 1.0 M sorbitol and 2.5% agar kept at 45° C.) and the appropriate selective medium containing 0.9M sorbitol and 3% glucose. Transformants can be recovered after incubation for 3-4 days at 30° C.

Li+ Transformation

This method involves treatment of yeast cells with specific monovalent alkali cations (Na+, K+, Rb+, Cs+and Li+) are used in combination with PEG to stimulate plasmid DNA uptake by intact yeast cells. Ito et. al in 1983 J. Bacteriology “Transformation of Intact yeast cells treated with alkali cations” 353: 163-168. This was followed by a 5 minute heat shock after which the cells were plated on selective medium. Best results are with Li Acetate (LiAc). The addition of a sonicated carrier DNA may be used to increase efficiency and the addition of a single stranded DNA or RNA to the reaction is used to optimize the reaction. Two vectors carrying different selectable marker genes may be used to knockout two different genes in a single transformation reaction or to looks for nonselective gene disruption using co-transformation with a selective plasmid. Below is a standard protocol for the LiAc/ssDNA/PEG protocol which has been shown to woke with most laboratory strains and is suitable for high—efficiency transformation of plasmid libraries for applications such as the yeast two-hybrid system.

Sample LiAc/ss DNA/PEG Protocol

1. Cells are grown overnight in 2× YPAD, resuspended at 5×10⁶ cells/mL in warm 2× YPAD and regrown for two cell divisions to 2×10⁷ cells/mL.

2. The cells are harvested by centrifugation at 3000× g for 5 min, washed twice in sterile distilled water, and resuspended in sterile distilled water at 10⁹ cells/mL.

3. Samples are 10⁸ cells are transferred to 1.5 mL microcentrifuge tubes, the cells are pelleted, and the supernatant are discarded.

4. The pellets are resuspended in 360 μL transformation mixture (240 μl 50% PEG 3500 (w/v), 36 μL 1.0 M LiAc, 50 μL 2.0 mg/mL single-stranded carrier DNA, 0.1-10 μg plasmid DNA plus water to 34 μL).

5. The cells in transformation mixture are incubated at 42° C. for 40 min. The cells are pelleted in microcentrifuge, and the transformation mixture is removed.

6. The cell pellet is gently resuspended in 1 mL sterile water, and samples are plated onto selective medium.

Electroporation

Electroporation, the use of electronic pulses to result in the formation of transient pulse in the cell membrane is widely used in transformation of plant and animal cells. It has also been used with yeast spheroplasts as well as intact yeast cells. Karube 1985 FEBS lett 182:90-94; Hashimoto 1985; Appl. Microbiol. Biotechnol 21:336-339. Electroporation has also been combined with PEG, as well as the LiAc/ssDNA/PEG method. A standard electroporation protocol is reproduced below:

Table 3. Sample Electroporation Protocol

1. Cells are grown in YPD to a density of 1×10⁷ cells/mL.

2. The cells are harvested by centrifugation (1500× g for 5 min), resuspended at 1×10⁹ cells/mL in 25 mM DTT (made in YPD medium, 20 mM HEPES, pH 8.0) and incubated for 10 min at 30° C.

3. The cells are then washed twice with EB (10 mM Tris-HC1, pH 7.5, 270 mM sucrose, 1 mM MgCl₂) and resuspended at 1×10⁹ cells/mL in EB.

4. Samples of 48 μL are mixed with 2 μL plasmid DNA and delivered between the electrodes of a square pulse generator CNRS cell electropulsator.

5. The cells are pulsed with a field strength of 1.74 kV/cm and a pulse length of 15 ms.

6. One milliliter of prewarmed 30° C. YPD is added immediately, and the suspension is incubated for 1 h at 30° C. The cells are then pelleted in a microcentrifuge resuspended in SD medium and plated onto the appropriate medium and incubated.

Yeast transformation has also been accomplished with glass beads, Costanzo et al, 1988. Genetics 120:667-670; as well as with biolisitics, Klein, et al 1987. Nature 327:7073.

The spheroplast, lithium cation and electroporation have been applied to most yeast species including, S. pompe, Candida albicans, Pichia pastoris, Hansenula polymorpha, Klyveromyces spp, Yamadazyma ohmeri, Yarrowia lipolytica, and Schwanniomyces occidentalis.

Additional information on yeast transformation may be found in the following: Gietz, et al., “Genetic Transformation of Yeast” BioTechniques 30:816-831 (April 2001); and Wang et al, “Transformation Systems of non-Saccharomyces Yeasts” Crit. Rev. Biotechnol. 2001; 21(3):177218.

It is often desirable to have the DNA sequence in homozygous state, which may require more than one transformation event to create a cell line; requiring transformation with a first and second recombinant DNA molecule both of which encode the same gene product. It is further contemplated in some of the embodiments of the process of the invention that a yeast cell be transformed with a recombinant DNA molecule containing at least two DNA sequences or be transformed with more than one recombinant DNA molecule. The DNA sequences or recombinant DNA molecules in such embodiments may be physically linked, by being in the same vector, or physically separate on different vectors. A cell may be simultaneously transformed with more than one vector provided that each vector has a unique selection marker gene. Alternatively, a cell may be transformed with more than one vector sequentially allowing an intermediate regeneration step after transformation with the first vector. Further, it may be possible to perform a sexual cross between individual yeast cells or yeast lines containing different DNA sequences or recombinant DNA molecules preferably the DNA sequences or the recombinant molecules are linked or located on the same chromosome, and then selecting from the progeny of the cross, yeast containing both DNA sequences or recombinant DNA molecules.

Expression of recombinant DNA molecules containing the DNA sequences and promoters described herein in transformed yeast cells may be monitored using northern blot techniques and/or Southern blot techniques known to those of skill in the art.

The regenerated yeast are transferred to standard growing media (e.g., solid or liquid nutrient media, grain, vermiculite, compost, peat, wood, wood sawdust, straw, etc.) and grown or cultivated in a manner known to those practiced in the art.

After the polynucleotide is stably incorporated into regenerated transgenic yeast, it can be transferred to other yeast by sexual crossing. Any of a number of standard techniques can be used, depending upon the species to be multiplied.

It may be useful to generate a number of individual transformed yeast with any recombinant construct in order to recover yeast free from any positional effects. It may also be preferable to select yeast that contain more than one copy of the introduced recombinant DNA molecule such that high levels of expression of the recombinant molecule are obtained.

As indicated above, it may be desirable to produce yeast lines that are homozygous for a particular gene if possible in the particular species. In some species this is accomplished by the use monosporous cultures. By using these techniques, it is possible to produce a haploid line that carries the inserted gene and then to double the chromosome number either spontaneously or by the use of colchicine. This gives rise to a yeast strain that is homozygous for the inserted gene, which can be easily assayed for if the inserted gene carries with it a suitable selection marker gene for detection of yeast carrying that gene.

The following examples is intended to further illustrate the invention and are not limit the invention in any way. The examples and discussion herein may specifically reference S. cerevisiae, however the teachings herein are equally applicable to any other yeast species. All references cited herein are hereby incorporated in their entirety by reference.

### EXAMPLES

### Example 1

Proteins destined for the secretory pathway first pass through the membranes of the endoplasmic reticulum (ER). To enter the lumen, they traverse a proteinaceous pore termed the “translocon” (Johnson and van Waes, 1999). Nascent soluble proteins are released into the lumen, whereas membrane proteins are integrated into the ER membrane. Since these proteins are translocated in an unfolded state, assembly into their native conformations occurs as a subsequent step in the ER. For this, the organelle maintains an inventory of raw materials, enzymes, and chaperones needed for proper protein folding and modification. Due to the localized nature of these functions, a mechanism termed “ER quality control” prevents transport of newly synthesized polypeptides to their sites of function until they reach their native conformation (Ellgaard et al., 1999).

The quality control mechanism also plays important roles when proteins fail to fold. Misfolded proteins are directed to a degradative pathway termed ER-associated protein degradation (ERAD) (Sommer and Wolf, 1997; Brodsky and McCracken, 1999). In this pathway, degradation does not occur in the lumen of the ER. Instead, proteins are transported back to the cytosol via the same translocon complex used for import (Wiertz et al., 1996; Pilon et al., 1997; Plemper et al., 1997; Zhou and Schekman, 1999). The process, termed retrotranslocation or dislocation, is usually coupled to ubiquitination, a requisite covalent modification of the substrate for degradation (Biederer et al., 1997). Ubiquitination takes place on the cytosolic surface of the ER, since the E2 and E3 enzymes Ubc7p and Hrdlp/Der3p, respectively, are localized there and may be positioned adjacent to the translocon (Hiller et al., 1996; Bordallo et al., 1998; Bays et al., 2001). Once marked, these proteins are rapidly degraded by the cytosolic 26S proteasome (Hiller et al., 1996).

Although much is known about the fate of ERAD substrates near the point of degradation, much less is understood regarding how they are recognized, retained, and targeted to the translocation/ubiquitination machinery. One model emerged that nascent polypeptides remain partially in the translocon after import. The polypeptide can only be released upon folding, whereas misfolded proteins are retrotranslocated via the same pore. The hypothesis was appealing, since it provided for a simple mechanists for retention and degradation. The model was brought into question when a well-established yeast soluble ERAD substrate, a mutant version of carboxypeptidase Y called CPY*, was shown to be translocated completely across the membrane (Plemper et al., 1999).

In mammalian cells, a mutant version of the well-characterized vesicular stomatitis virus G (VSV-G) protein, ts045, was observed to be localized to the ER of cells shifted to 39.5° C., a temperature that causes it to misfold (Kreis and Lodish, 1986). An elegant study using VSV-G ts045 fused to the green fluorescent protein provided direct evidence of an ER retention mechanism. Using photobleaching experiments in live cells, the integral membrane protein was shown to move freely in the plane of the membrane but did not leave the ER (Nehls et al., 2000). In cells overexpressing VSV-G ts045 through prolonged incubation at the restrictive temperature, a fraction of the protein escapes the ER and gets transported to the Golgi and retrieved (Hammond and Helenius, 1994). Although these earlier experiments were performed under more extreme conditions, they left open the possibility of a recycling mechanism for misfolded proteins. In yeast, the mechanism is less clear, but the efficient degradation of mutant versions of Ste6p and Yorlp integral membrane proteins in absence of ER-to-Golgi transport seems to support the mammalian view (Loayza et al., 1998; Katzmann et al., 1999).

A common quality control mechanism for both misfolded soluble and membrane proteins presents a spatial problem, since these two classes may occupy distinct regions of the ER (that is, luminal versus membrane). Therefore, it is plausible that different recognition and targeting mechanisms exist to direct the proteins into the degradation pathway. In this view, the ubiquitin/proteasome pathway used by both misfolded soluble and membrane proteins can be thought of either as an endpoint for an ER retention mechanism or a point of convergence for distinct mechanisms.

In this study, applicants examined the fate of several quality control substrates subject to ERAD-specific degradation in the budding yeast Saccharomyces cerevisiae. The coexistence of retention and retrieval mechanisms that define distinct classes of quality control substrates have been demonstrated. For both pathways, a sorting step occurs in the ER whereby substrates of the retrieval pathway are packaged into COPII transport vesicles, whereas those to be retained are excluded. Furthermore, by using a genetic approach applicants isolated mutants dissecting the two pathways. A mutant loss of function allele of the gene BSTI called per17-1 (any loss of function mutation would have a similar effect) prevented the ER-to-Golgi transport of misfolded proteins while preserving the transport of most normal proteins. In per17-1 cells, quality control is disrupted at an early step of the retrieval pathway as observed by the accumulation and stabilization of misfolded proteins in subcompartments associated with the ER.

KHN is a Misfolded Protein Retrieved from the Golgi Apparatus for ERAD

Viral membrane proteins are excellent models to study protein folding and ER quality control (Gething et al., 1986; Machamer et al., 1990; Hammond and Helenius, 1994). To better understand quality control mechanisms, applicants sought to combine their advantages with the facile genetics of the budding yeast S. cerevisiae, although the teachings herein are equally applicable to any fungi species. The simian virus 5 hemagglutinin neuraminidase (HN), was selected since its folding state can be monitored using established methods (Ng et al., 1989). To express HN, the HN signal/anchor domain was replaced with the cleavable signal sequence from the yeast Kar2 protein and placed the fusion construct downstream of the moderate yeast PRO (CPY) promoter. This was done to bypass the poor utilization of the endogenous signal/anchor domain in yeast. The resulting protein, designated KHN, is similar to a soluble version of HN characterized previously in mammalian cells (Parks and Lamb, 1990).

Applicants monitored the expression of KHN by metabolic pulse-chase analysis and made an unexpected observation. As shown in FIG. 6A, KHN is lost rapidly after a 30-min chase and is nearly undetectable by 60 min. Since proteins from both cells and medium were combined for immunoprecipitation, secretion of KHN was ruled out to account for the loss. Alternatively, as a foreign protein KHN may fail to properly fold and be subject to quality control mechanisms leading to its degradation. Consistent with this notion, KHN fails to form disulfide-linked dimers and is not reactive to conformation-dependent anti-HN monoclonal antibodies. In a strain deleted of CUD, a gene required for ubiquitination of proteins destined for ERAD (Biederer et al., 1997), KHN appeared to be stabilized during the same time course (FIG. 6 A, middle). Applicants confirmed KHN as a bona fide ERAD substrate, since it is stabilized by multiple ERAD-specific mutants (see below). Interestingly, stabilization of KHN enhanced an unexpected characteristic for an ERAD substrate, that is, a time-dependent decrease in gel mobility (FIG. 6A, p1 and p2). Applicants next explored the nature of the altered forms.

Stepwise increases in molecular weight are commonly observed during the maturation of many yeast secretory pathway proteins. The increase is due to elaboration of carbohydrates attached initially in the ER (Herscovics and Orlean, 1993). The delay reflects the time needed to transport nascent polypeptides to the Golgi apparatus where the modifying enzymes reside (Gemmill and Trimble, 1999; StrahlBolsinger et al., 1999). With this in mind, the observed modification raised the intriguing possibility that KHN is transported to the Golgi and retrieved to the ER for degradation. Applicants addressed this possibility by first determining whether the shifts are actually due to carbohydrate modification. Endoglycosidase H digestion was used to remove N-linked carbohydrates from KHN. If the gel mobility shifts were due solely to modification of N-linked sugars, all forms of KHN after endoglycosidase H treatment would migrate equally. As shown in FIG. 6A (right), removal of N-linked sugars did not eliminate the mobility differences. Applicants next tested for O-linked carbohydrates by using mutants specifically defective at the first step of O-mannosylation. O-mannosylation begins in the ER with the transfer of a single mannose residue from Man-P-dolichol to the polypeptide. Enzymes of the protein mannosyltransferase (PMT) family catalyze this reaction. Strains deleted of individual PMT genes exhibit substrate-specific defects in glycosylation, reflecting the nonredundant nature of these genes (Gentzsch and Tanner, 1996). Applicants expressed KHN in strains singly deleted of each PMT family member (PMT1-PMT6). As shown in FIG. 6B, strains deleted of PMTI and PMT2 prevented KHN mobility shifts such that p1 remained the predominant form that is degraded eventually. These data show that KHN O-glycosylation is dependent on PMT1 and PMT2 whose products were shown previously to work together as a complex (Gentzsch et al., 1995). The particular protein specific KHN processing was unaffected in strains singly deleted of PMT3-PMT6 which is likely a result of substrate specificity for this protein. Other PMT will likely work with other proteins.

Proteins O-mannosylated in the ER are usually modified through lengthening of the carbohydrates in the Golgi (Lussier et al., 1997). To test whether the KHN gel mobility shift is due to post-ER processing, Applicants expressed KHN in the well-characterized ER to-Golgi transport mutants sec12-4 and sec18-l (Eakle et al., 1988; Nakano et al., 1988; Barlowe and Schekman, 1993). When transport is blocked in these strains, KHN remains in the p1 form over an extended time course (FIG. 6C). This is consistent with formation of the p2 form in the Golgi apparatus where the modifying enzymes reside. From these data, we designate the ER form as p1 and the Golgi form as p2. Interestingly, the turnover of KHN appears to be impaired in these mutants, suggesting that transport out of the ER may be a required step for degradation. Unfortunately, nonspecific immunoprecipitation of proteins overlapping the p1 and p2 forms made the kinetics of KHN turnover difficult to measure. Thus, the extent of the stabilization was inconclusive from these experiments.

To accurately measure the kinetics of KHN turnover, a modified version was constructed bearing a COOH-terminal triple HA epitope tag (KHNₜ). When using the anti-HA monoclonal antibody, immunoprecipitations of KHNₜ were free of background, and the yields were otherwise indistinguishable from experiments using the anti-HN polyclonal antisera (FIG. 7A). KHNₜ is modified and degraded similarly to KHN except that the rate of turnover seems to be reduced slightly (FIG. 7A, top, compared with FIG. 6A, left). Although preliminary results suggested that KHN might be a substrate of the ERAD pathway, its transport to the Golgi raised the possibility that a fraction might continue forward and degrade in the vacuole (the yeast equivalent of lysosomes). This was ruled out when KHNₜ was degraded similarly to wild type in a mutant deficient in functional vacuolar proteases (FIG. 7, A and B, Δpep4). To establish firmly that KHN is a substrate of ERAD, Applicants measured the stability of KHNₜ in several mutants defective specifically in the pathway. As shown in FIG. 7, when KHNₜ is expressed in strains deleted of the CUE1 (role in ubiquitination by anchoring Ubc7p to the ER membrane), DER1 (encodes an ER membrane protein required for ERAD), or HRD1/DER3 (encodes an ER-localized E3 ubiquitin ligase) genes, its degradation is impaired to the extent similar to other established ERAD substrates (Hampton et al., 1996; Knop et al., 1996; Biederer et al., 1997; Bordallo et al., 1998). Western blot analysis shows the steady-state accumulation of higher molecular weight (p2) forms of KHNₜ in each of the mutants, confirming that it is these species that are preferentially degraded in wild-type cells (FIG. 7C).

Misfolded proteins accumulate in the ER of cells defective for ERAD functions (Knop et al., 1996; Loayza et al., 1998). Since KHNₜ is transported to the Golgi before degradation, the question of where it accumulates when ERAD is disrupted was raised. By performing indirect immunofluorescence, Applicants found that KHN, also accumulates in the ER of ERAD mutant cells as shown by its colocalization with the ER marker BiP (FIG. 7D). These data show that KHNₜ behaves similarly to some established ERAD substrates and point to the possibility of a retrieval pathway for its degradation.

Two Distinct Mechanisms for the Quality Control of Proteins Destined for ERAD

The expression of KHN in ER-to-Golgi mutants led to an unexpected observation-transport may be an obligatory step for its degradation. This was surprising since other ERAD substrates, including mutant Ste6p and Yorlp, were observed to degrade normally under the same conditions Loayza et al., 1998; Katzmann et al., 1999). The apparent contradiction could be resolved if different mechanisms exist to target aberrant proteins for degradation: a static (nonrecycling) ER retention mechanism for proteins like Ste6p and Yorlp (both integral membrane proteins) and a transport and retrieval mechanism for others like KHN. To test this possibility, Applicants applied complementary in vivo and in vitro approaches to assess the fate of substrates before degradation.

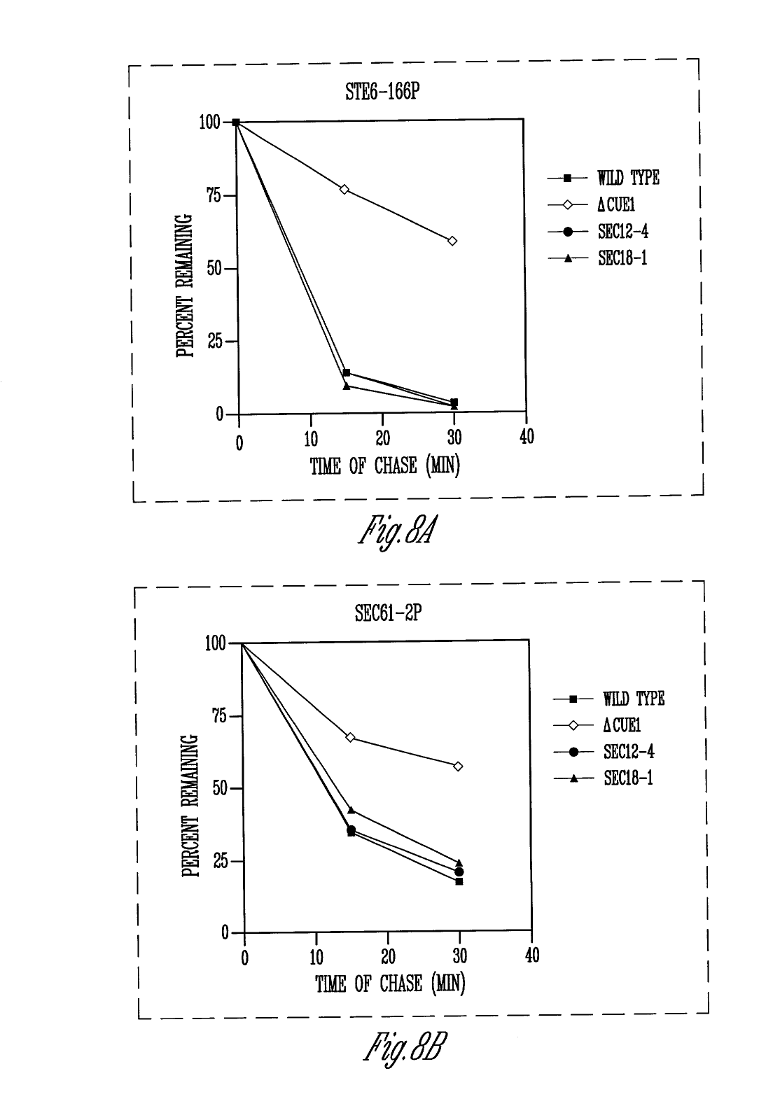

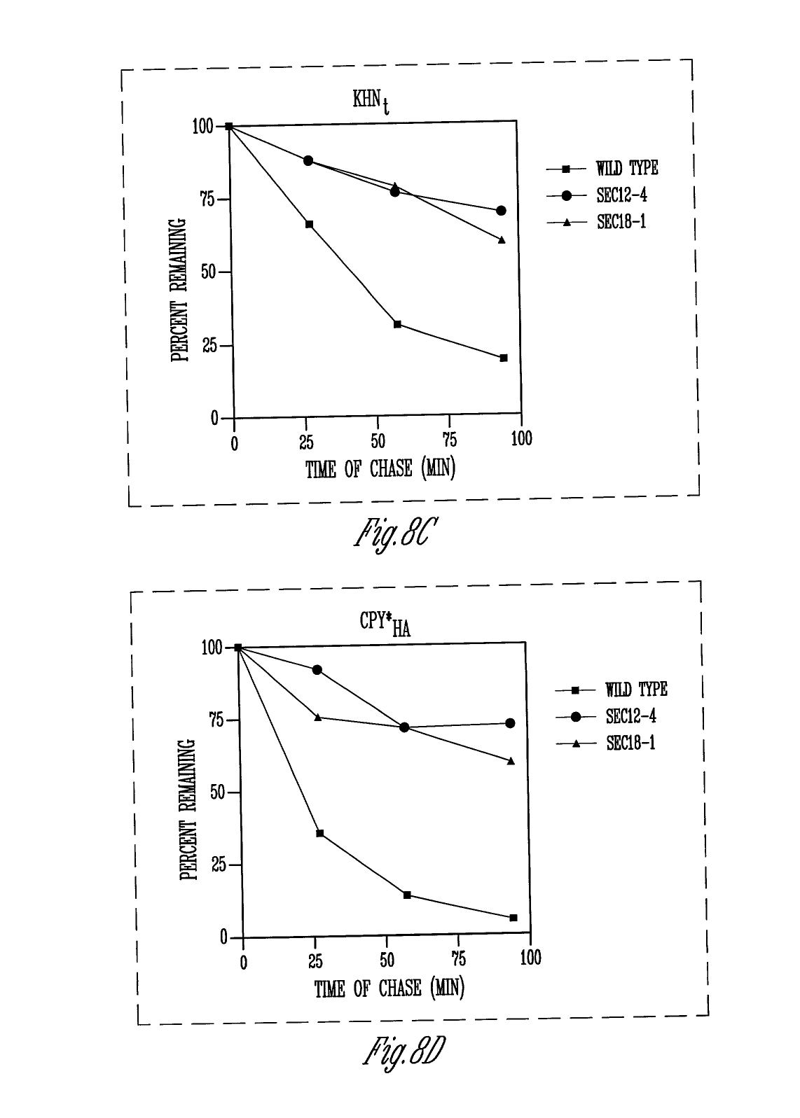

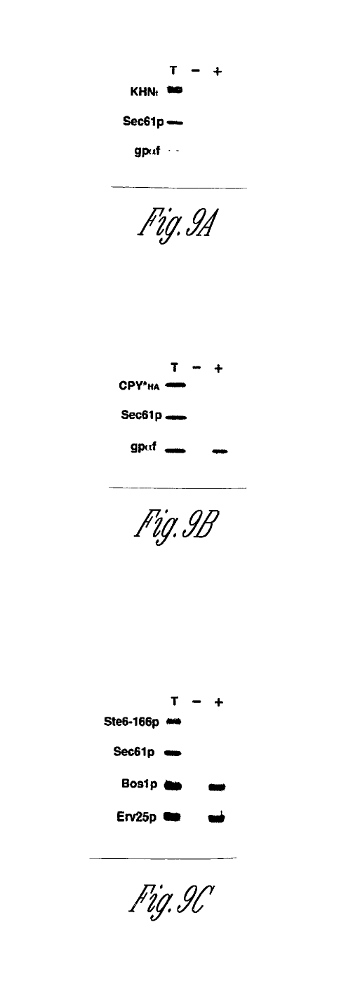

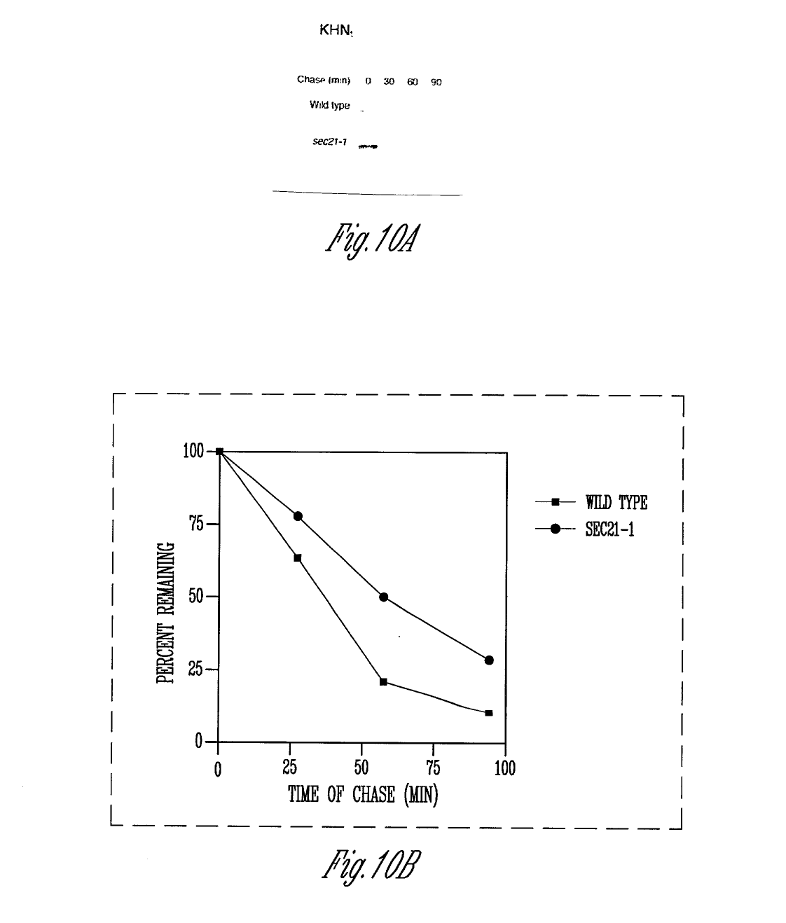

First, the effect of preventing ER-to-Golgi transport was examined. It was reported that the ERAD substrate Ste6-166p is degraded in a sec18 mutant, suggesting that ERAD functions normally even if transport is blocked (Loayza et al., 1998). Applicants confirmed the observation in both sec12 and sec18 cells by finding the stability of Stet-166p is identical to wild type (FIG. 8A). As a control, Applicants showed that an ERAD defective mutant stabilizes Ste6-166p under the same conditions (FIG. 8A). Applicants also analyzed Sec61-2p, another membrane protein subject to ERAD (Sommer and Jentsch, 1993; Biederer et al., 1996). Since Sec61p itself plays a role in ERAD, Sec61-2p was expressed ectopically and distinguished from wild type with an HA epitope tag. As with Ste6-166p, Sec61-2p is degraded normally under the restrictive conditions in each strain (FIG. 8B). By contrast, the degradation of KHNₜ was strongly impaired (FIG. 8C). Since core ERAD functions are normal in these strains, the defect is likely a consequence of perturbing the KHNₜ trafficking pattern that precedes degradation. The question of whether this requirement is unique to KHNₜ or reflects a more general feature of ER quality control was raised. For this, Applicants examined an HA epitope-tagged version of another well-characterized soluble substrate, CPY* (Finger et al., 1993). Although it is well established that CPY*_{HA} uses the core ERAD machinery, it was unclear whether it is retained or undergoes a retrieval cycle. As shown in FIG. 8D, CPY*_{HA} is stabilized strongly in both sec12 and sec18 mutants, suggesting that it too is dependent on the vesicular transport pathway. However, this was surprising, since it was reported previously that CPY* is degraded in a sec18 mutant (Finger et al., 1993). There, the degradation was most pronounced after a long chase period of 3 h. Applicants also observed some degradation in the transport mutants so we might expect a substantial fraction of the substrate to be degraded if we applied a similarly extended chase.

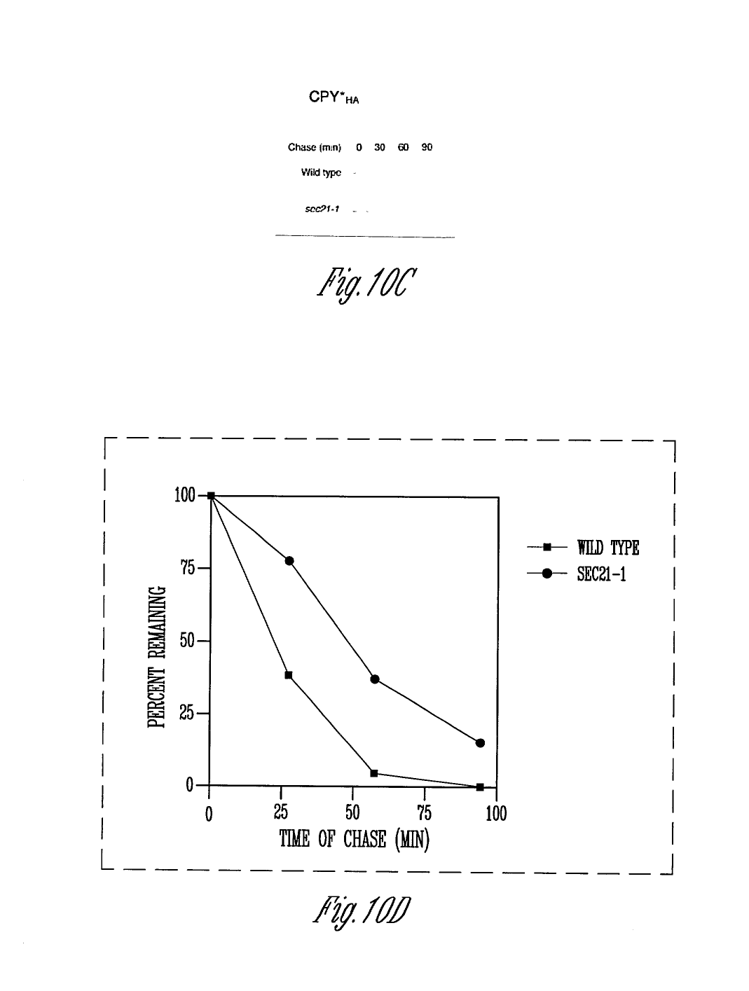

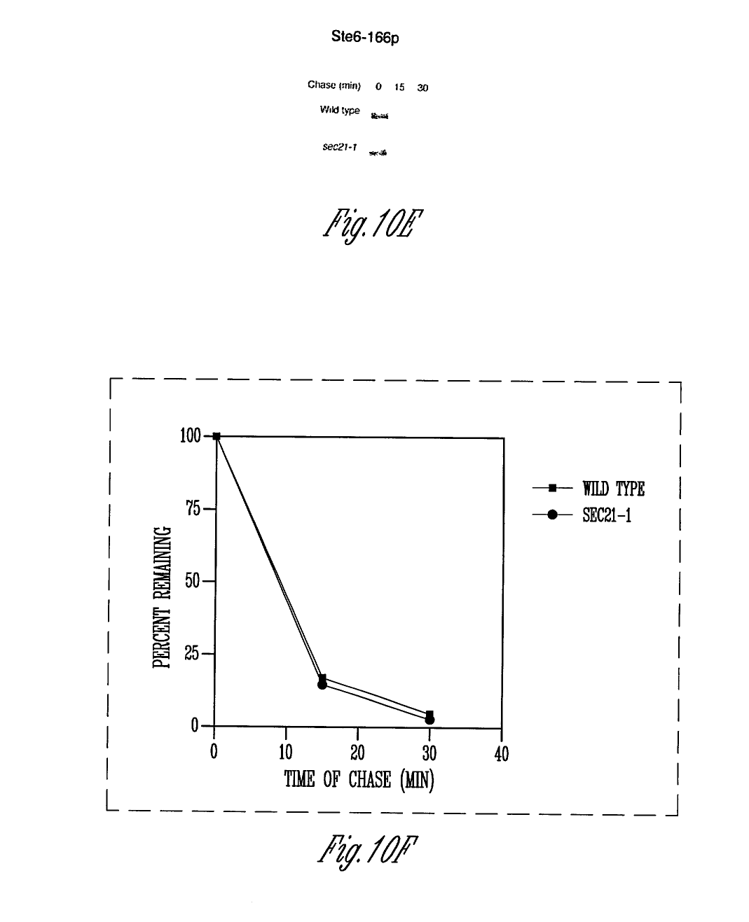

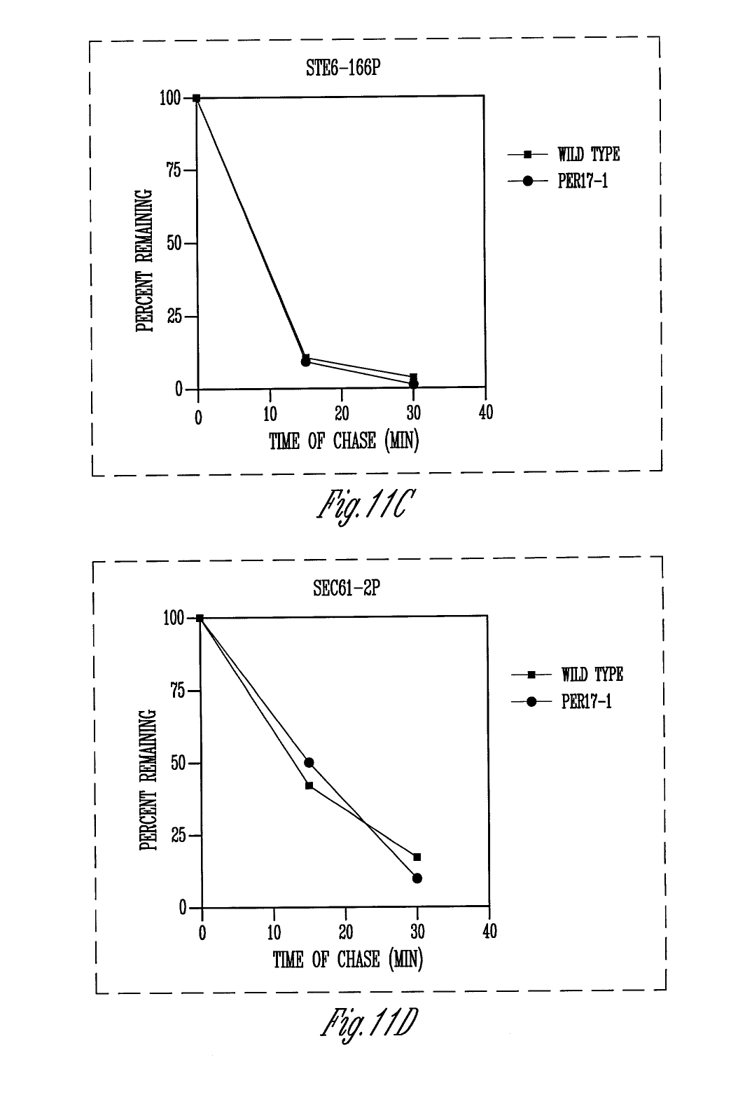

The data suggest two classes of ERAD substrates, one uses the vesicular trafficking machinery for quality control and the other depends on static ER retention. This distinction predicts that sorting takes place in the ER to segregate misfolded proteins to be transported from those to be retained. The differences in degradation rates in the sec12 and sec18 mutants provided only suggestive evidence and did not rule out the possibility of indirect effects. To test this in vitro assays that reproduce COPII-coated vesicle budding and cargo selection from ER membranes were performed (Barlowe et al., 1994). For these experiments, microsomes were prepared from wild-type strains expressing KHNₜ, CPY*_{HA}, and Ste6-166p. COPII budded vesicles from these microsomes were isolated, and the level of individual proteins packaged into vesicles were monitored by immunoblots (FIG. 9). The efficiency of incorporation for each protein was calculated as a percentage of the total by densitometry. For KHNₜ and CPY*_{HA}, Applicants found both proteins packaged into COPII vesicles at 1-2%, whereas the negative control Sec61p was not packaged. Although the amount of misfolded proteins packaged in COPII vesicles is less relative to other secretory proteins, it is consistent with the slower transport of KHNₜ compared with other cargo proteins (see FIG. 11B). For the analysis of KHNₜ and CPY*_{HA} by this method, membranes were treated with trypsin to ensure detection of protease-protected luminal species. These data provide independent confirmation that a subset of proteins destined for ERAD are first exported from the ER using the standard membrane trafficking machinery.

Next, Applicants examined Ste6-166p. There already exists evidence that Ste6-166p is targeted for degradation using an ER retention mechanism (Loayza et al., 1998). However, the nature of the retention was unclear. the ER vesicle budding assay was applied to Ste6-166p, it remained exclusively in ER membranes even as other integral membrane proteins were incorporated efficiently into COPII-coated vesicles (FIG. 9C). These data show that the plasma membrane protein Ste6p, when misfolded, is retained in the ER by exclusion from transport vesicles. Together, these results reveal a novel facet of ER quality control. As part of its surveillance mechanism, the cell sorts misfolded proteins for ER retention or transport.

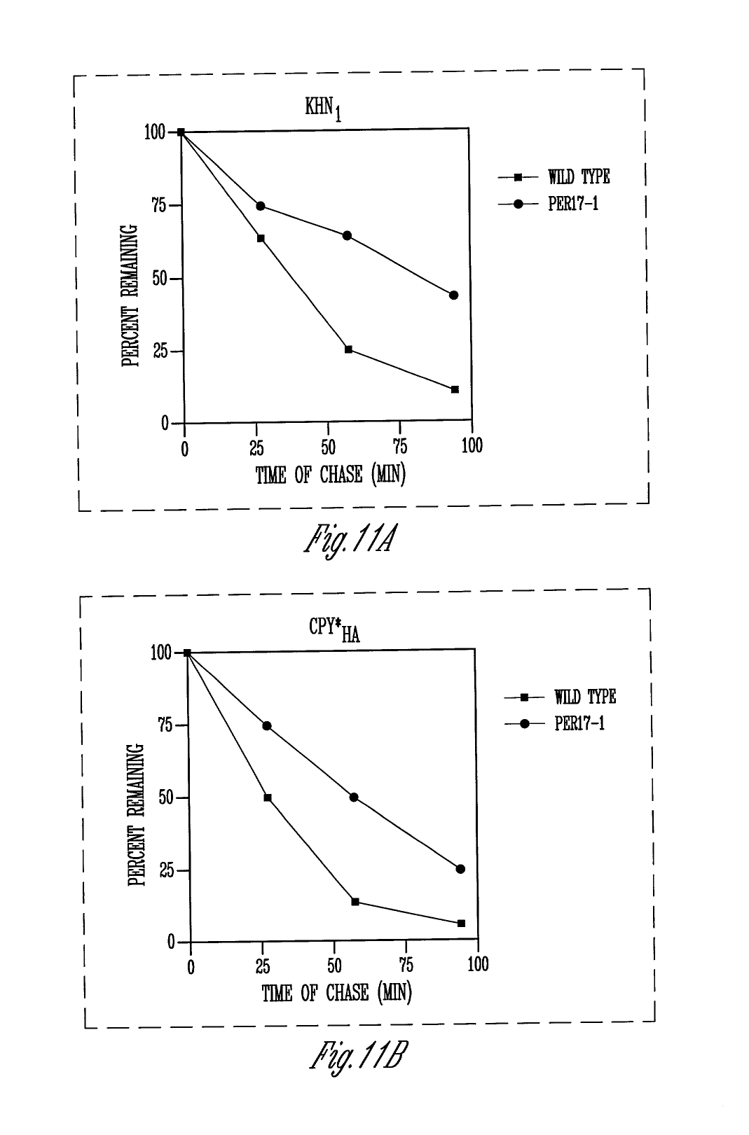

After transport to the Golgi, degradation of KHNₜ and CPY*_{HA} by ERAD would require retrograde trafficking to the ER. The reverse flow of membranes and proteins from the Golgi is driven by the formation of coated vesicles of the COPI class. To examine whether the trafficking of misfolded proteins use COPI-coated vesicles, applicants expressed KHN, and CPY*_{HA} in the γ-COP mutant sec21-1 and measured their turnover. At the permissive temperature of 30° C., where forward transport is unaffected and retrograde transport is little affected (Letourneur et al., 1994), a small but reproducible delay in KHNₜ and CPY*_{HA} degradation was observed. However, at the semipermissive temperature of 33° C., which partially disrupts retrograde transport with only a minor delay in forward transport (Letourneur et al., 1994), degradation of both proteins is inhibited (FIG. 10, A and B). The proficiency of forward transport was confirmed by analyzing endogenous CPY (unpublished data) and the formation of the KHN, p2 Golgi form (FIG. 10A). Indirect effects on ERAD function were ruled out, since Ste6-166p degradation is normal in sec21-1 cells (FIG. 10C). Taken together, these data demonstrate that misfolded proteins are sorted for ER retention or transport and retrieval from the Golgi. Ultimately, both pathways converge in the ER for degradation by the ERAD pathway.

A Gene Required for ER Quality Control Early in the Retrieval Pathway

Recent studies have demonstrated that some cargo proteins leaving the ER are actively sorted into transport vesicles (Muniz et al., 2001). Although the molecular mechanisms of these sorting events are not well understood, specific genes have been implicated for the transport of just a subset of proteins (Belden and Barlowe, 1996; Muniz et al., 2000). Since KHNₜ and CPY*_{HA} may represent a new class of cargo proteins, the question of whether dedicated factors function to sort and package misfolded proteins into transport vesicles was raised. To address this question, Applicants employed a genetic approach. If such factors exist, we reasoned that their loss of function would cause the retention and stabilization of misfolded proteins normally transported out of the ER. Applicants reported previously a genetic screen based on synthetic lethality with the unfolded protein response pathway as a powerful means of identifying genes associated with ER quality control (Ng et al., 2000). As the original screen was far from exhausted, the scope was expanded with the intent of dissecting the ER retention and recycling mechanisms of quality control. Applicants thus discovered of a gene needed for the anterograde transport of misfolded proteins in the retrieval pathway.

Starting with a pool of 152 recessive protein processing in the ER (per) mutants, those exhibiting general processing defects of normal proteins including glycosylation and transport were excluded (Ng et al., 2000). Of the remaining 107, ERAD activity was analyzed by measuring the stability of CPY*_{HA} and Sec61-2p as described in the legend to FIG. 8. Applicants analyzed the stability and processing of KHNT. For one mutant, per17-1, (loss of function) both KHNₜ and CPY*_{HA} are defective for degradation (FIG. 11A). However, unlike other ERAD mutants (FIG. 7), KHNₜ remains in the ER p1 form in per17-1 cells consistent with a transport block to the Golgi (FIG. 11B, top). Gas lp (FIG. 11B, bottom) and chitinase carbohydrate processing in per17-1 cells is normal and serves to control for functional O-mannosylation and modification in per17-1 cells (Nuoffer et al., 1991; Gentzsch and Tanner, 1996). This shows that the prevalence of the KHNₜ p1 form reflects a transport defect rather than an indirect effect on glycosylation. Interestingly, transport of folded cargo proteins showed differential effects. CPY transport was similar to wild type, whereas Gas1p was slower than normal (FIG. 11B). Since Gas1p is anchored in the membrane, applicants examined two additional integral membrane cargo proteins, carboxypeptidase S (CPS) and alkaline phosphatase (ALP) (Cowles et al., 1997; Spormann et al., 1992). As shown in FIG. 11C, both proteins are transported indistinguishably to wild type, confirming that the per17-1 mutation does not cause general defects in ER-to-Golgi transport.

The data suggest that the per17-1 mutation inhibits degradation by failing to promote the transport of misfolded proteins destined for the retrieval pathway. To reinforce this view, we analyzed the fate of ERAD substrates that are sorted for ER retention. If PER17 plays such a distinct role in ER quality control, the retention pathway is expected to be functional and these substrates to turn over normally in per17-1 cells. As shown in FIG. 11A (bottom), Ste6-166p and Sec61-2p are degraded with wild-type kinetics in per17-1 cells. These data show that the per17-1 allele is specific to the recycling pathway and validates our genetic strategy. Although these data are similar to those obtained using the sec12 and sec18 mutants, they extend the evidence that transport is an important step for degradation, since the per17-1 transport block affects misfolded soluble proteins while leaving the transport of several normal cargo proteins intact.

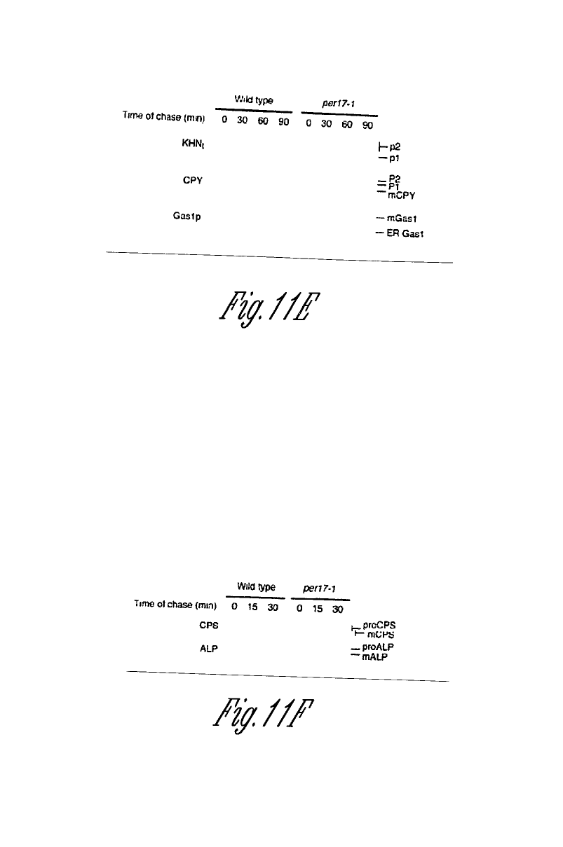

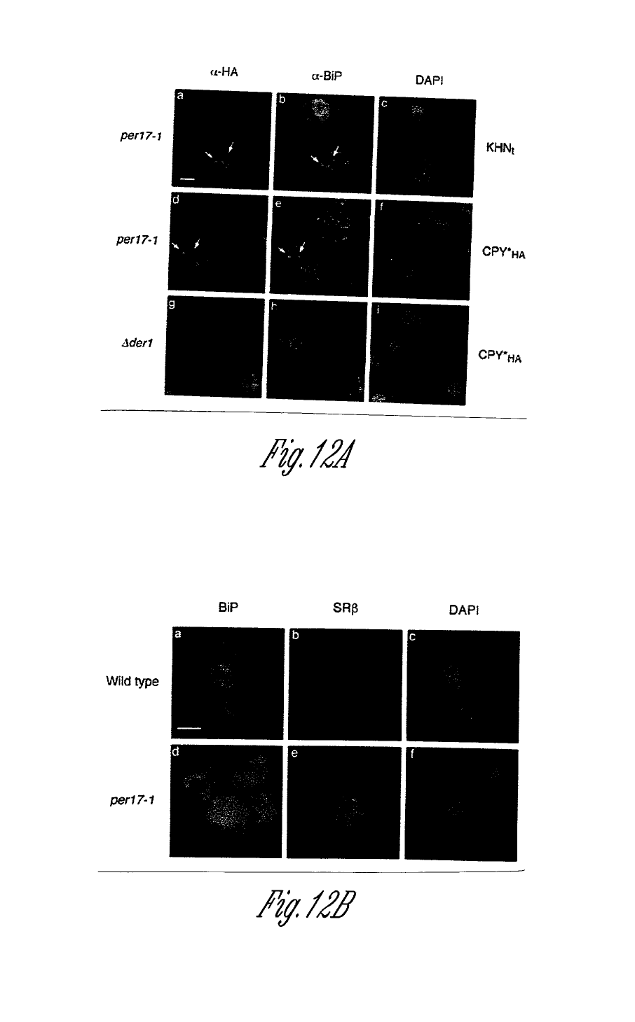

To better understand the nature of the per17-1 transport block, Applicants performed indirect immunofluorescence to localize KHNₜ and CPY*_{HA} stabilized in per17-1 cells. As shown in FIG. 12, both KHNₜ and CPY*_{HA} are concentrated within punctate structures throughout the cell. This differs from transport competent ERAD mutants, since they accumulate these substrates diffusely throughout the ER (Knop et al., 1996; FIG. 7D). Interestingly, the punctate distribution is reminiscent of the pattern observed for cargo proteins blocked for transport in sec12 mutant cells (Nishikawa et al., 1994). In those cells, the ER chaperone BiP colocalized with cargo proteins at discrete sites within the ER. Since the misfolded proteins are similarly blocked for transport, we also examined the distribution of BiP in the per17-1 cells. As shown in FIG. 12, BiP was found in the same punctate structures as KHNₜ and CPY*_{HA} (FIG. 12A, b and e) Although BiP is widely used as a marker for ER morphology, Applicants questioned whether the pattern reflected subdomains of the ER as the case in sec12 cells or a general reorganization of ER membranes. To address this, an alternative ER marker, was chosen, the signal recognition particle receptor β subunit (SRβ). SRβ is an integral membrane protein that is distributed throughout the ER (Ogg et al., 1998). As shown in FIG. 12B, SRβ staining in per17-1 cells is similar to wild type, indicating that there are no gross changes in ER morphology (FIG. 12B, e). This is in good agreement with ultrastructural analysis performed with the same strains. In double-label experiments, the punctate structures are always coincident with the ER as defined by SRβ (FIG. 12B). These data show that misfolded proteins accumulate with BiP at distinct ER sites in per17-1 cells.

The identity of the PER17 gene was next determined. A yeast genomic library based on the centromeric YCp50 vector was transformed into the per17-1 mutant. By restoration of the sectoring phenotype, a complementing clone was obtained (Ng et al., 2000). Through deletion mapping, a single ORF encoding the BST1 (bypass of sec thirteen) was identified as the PER17 gene. BST1 encodes an ER integral membrane protein first cloned through genetic interaction with SEC13, a component of the COPII vesicle coat (Elrod-Erickson and Kaiser, 1996). Thus, BST1 is believed to play a role in ER-to-Golgi transport. However, its precise role was unknown in the art, since a BSTI gene deletion did not seem to affect the transport of two prototypic cargo proteins, CPY and invertase. The data suggest a novel function for BST1 in ER quality control. Since per17-1 and Δbst1 cells prevent the transport of misfolded but not most properly folded proteins, the data suggest a role in cargo protein sorting (FIG. 11B; unpublished data).

Discussion

A cellular surveillance system that monitors the folding state of nascent proteins in the ER was first observed nearly a quarter century ago. Those pioneering studies showed that viral membrane proteins, when misfolded, were not transported to the plasma membrane but retained at the site of synthesis (Gething et al., 1986; Kreis and Lodish, 1986). Subsequently, the phenomenon was appropriately termed “ER quality control” and led to the realization that several human diseases, including cystic fibrosis, owed their molecular basis to the retention and degradation of mutant proteins (Carrell and Gooptu, 1998; Kim and Arvan, 1998; Kopito and Ron, 2000). More recently, important strides have improved our understanding of ER quality control. Most notably, the degradation step, or ERAD, is now known to involve the retrotranslocation of substrates to the cytosol through the ER translocon pore Wiertz et al., 1996; Pilon et al., 1997; Plemper et al., 1997; Zhou and Schekman, 1999). During or after retrotranslocation, substrates are ubiquitinated and degraded by the 26S proteasome (Ward et al., 1995; Hiller et al., 1996). Despite these advances, the events upstream to ERAD remained unclear.

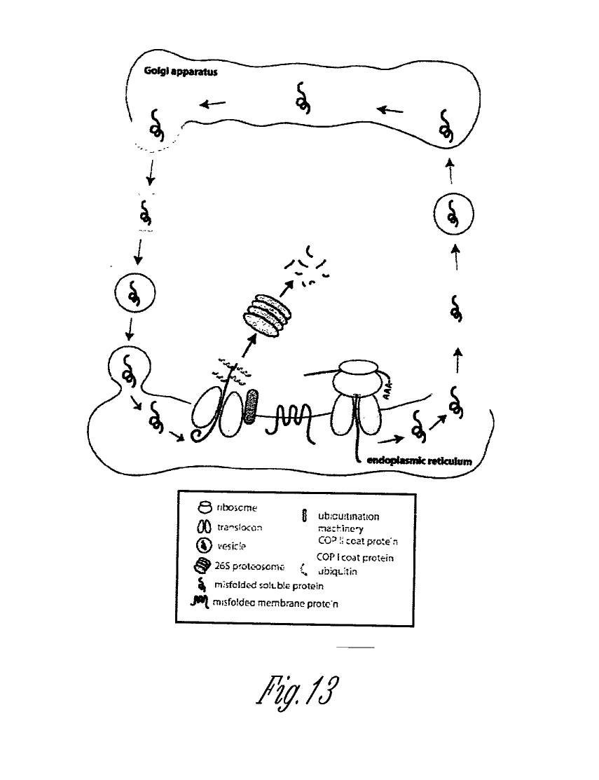

Applicants herein disclose the collaboration of two distinct mechanisms to assure the quality control of protein biosynthesis in the yeast secretory pathway. By combining biochemical and genetic approaches, the retention mechanism was reconfirmed while uncovering another that uses established ER to-Golgi vesicle transport and retrieval pathways (FIG. 13). Applicants disclosed direct evidence of ER-to-Golgi transport of misfolded proteins in vivo and in vitro and a requirement for retrograde transport.

Key to the approach was the characterization of KHN as a novel ERAD substrate. Unlike other misfolded proteins commonly studied, KHN allows the use of O-linked sugar modifications to monitor its transport (FIG. 6). The native HN protein is not O-glycosylated in mammalian cells so it seems likely that the modifications are due to promiscuous O-mannosylation that can occur when proteins misfold in yeast (Harty et al., 2001). The processing of these carbohydrates shows that most, if not all, of the protein uses a retrieval mechanism before ERAD. Furthermore, applicants found that disruption of either forward or retrograde transport compromised KHN degradation. The transport requirement is not peculiar, since the well-characterized substrate CPY* is affected similarly under all circumstances. Since substrates subject to retention are degraded normally in these mutants, the data strongly suggest that transport and retrieval are obligatory steps for efficient KHN and CPY* degradation.

An in vitro vesicle budding assay using purified components provided direct evidence that KHN and CPY* are packaged into COPII-coated vesicles, whereas Ste6-166p is excluded. These experiments were important, since the assay was established previously to reflect early events in ER-to-Golgi transport. Although the data serve to confirm and extend the in vivo experiments, they also reveal a novel ER sorting mechanism for misfolded proteins at or just before the formation of COPII vesicles. The retrieval pathway largely uses the standard vesicle transport machinery, but we do not know whether misfolded proteins occupy the same vesicles as folded cargo proteins. Recently, it was shown that different classes of folded cargo proteins occupy distinct vesicle populations (Shimoni et al., 2000; Muniz et al., 2001). Thus, it seems possible that misfolded proteins are sorted into specialized vesicles for transport to the Golgi.

Materials and Methods

Plasmids Used in this Study

Plasmids were constructed using standard cloning protocols (Sambrook et al., 1989). For pDN431 and pDN436, HA epitope-tagged CPY* expression vectors were described previously (Ng et al., 2000). For pSM1083 and pSM1346, HA epitope-agged Ste6-66p expression vectors were gifts from S. Michaelis Johns Hopkins University, Baltimore, Md.) (Loayza et al., 1998).

Construction of the HA Epitope-tagged Sec61-2p Expression Vector pDN1002

The promoter and coding sequences of sec61-2 were cloned from strain RSY533 (MATα, sec61-2, leu2, ade2, ura3, pep4-3) by amplification of genomic DNA using Vent polymerase (New England Biolabs, Inc.) performed according to manufacturer's protocol. Using the primers N782 (5′-CGAATCCGTCGTTCGTCACC3′) and N183 (5′-TTCCCATGGAATCAGAAAATCCTGG-3′), the amplified 2,016-bp fragment was digested with HindIII and NcoI, and the 1,931-bp fragment was purified. The purified fragment was ligated into pDN333 digested with the same enzymes. pDN333 was generated by inserting the HA-tagged insert from pDN280 (Ng et al., 1996) into pRS315 (Sikorski and Hieter, 1989). An Ncol site from N183 places the Sec61-2p coding sequence in frame with vector sequences encoding a single HA tag followed by ACTT terminator sequences.

Construction of KHN Expression Vectors pSM31, pSM56, pSM70, and pSM72

The KHN fusion gene was constructed by ligating the sequences encoding the first 45 amino acids of Kar2p (signal sequence and signal peptidase cleavage site) to the COOH-terminal 528 amino acids of the SV5 HN gene. Both fragments were amplified by PCR using Vent polymerase and inserted into pDN251 to generate pSM31. pDN251 is identical to the yeast expression vector pDN201 (Ng et al., 1996) except it contains the moderate PRC7 promoter in place of the TDH3 promoter. pSM70 is identical to pSM31 except for the addition of a triple HA epitope tag inserted in-frame to the COOH terminus of KHN. Sequences encoding the triple HA epitope tag were excised from pCS124 (a gift from C. Shamu, Harvard University, Cambridge, Mass.). pSM56 and pSM72 are similar to pSM31 and pSM70, respectively, except that the KHN gene sequences were subcloned into pRS315.

pES69 was constructed by inserting a NotI/KpnI fragment containing the gene for HA epitope-tagged SRβ from pS0459 (Ogg et al., 1998) into pRS426 (Sikorski and Hieter, 1989).

Strains and antibodies

Yeast strains used in this study are described in Table I. Anti-HA monoclonal antibody (HA.11) was purchased from BabCo. Anti-Kar2p antibody was provided by Peter Walter (University of California, San Francisco, Calif.). Anti-CPY antiserum was provided by Reid Gilmore (University of Massachusetts, Worcester, Mass.). Anti-Gas1p was a gift from Howard Riezman (University of Basel, Basel, Switzerland). Anti-ALP and anti-CPS antisera were gifts from Chris Burd and Scott Emr (University of California, San Diego, Calif.). Anti-HN antiserum was described previously (Ng et al., 1990). Secondary antibodies labeled with Alexa Fluor 488 or 546 were purchased from Molecular Probes, Inc.

Cell Labeling and Immunoprecipitation

Typically, 2 A₆₀₀ OD U of log phase cells were pelleted and resuspended in 1.0 ml of synthetic complete medium lacking methionine and cysteine. After 30 min of incubation at the appropriate temperature, cells were labeled with 480 μCi of Tran³⁵S-label (ICN Biomedicals). A chase was initiated by adding cold methionine/cysteine to a final concentration of 2 mM. The chase was initiated 30 s before the end of the pulse to exhaust intracellular pools of unincorporated label. Labeling/chase was terminated by the addition of trichloroacetic acid to 10%. Preparation of cell lysates, immunoprecipitation procedures, gel electrophoresis, and quantification of immunoprecipitated proteins were performed as described previously (Ng et al., 2000).

In vitro Budding Assays

Vesicle budding from the ER was reproduced in vitro by incubation of microsomes (Wuestehube and Schekman, 1992) with purified COPII proteins (Sar1p, Sec23p complex, and Sec13p complex) as described (Bariowe et al., 1994). Microsomes were prepared from cells expressing misfolded KHNₜ CPY′_{HA} and Ste6-166p (SMY248, WKY114 and SMY225). To measure incorporation of proteins into COPII vesicles, a 15-μl aliquot of the total budding reaction and 150 μl of a supernatant fluid containing budded vesicles were centrifugred at 100,000 g in a TLA100.3 rotor (Beck. Man Coulter) to collect membranes. The resulting membrane pellets were solubilized in 30 μl of SDS-PAGE sample buffer, and 10-15 μl were resolved on 12.5% polyacrylamide gels. For measurement of KHNₜ and CPY′ contained in COPII vesicles, membranes were treated with trypsin (100 μg/ml) for 10 min on ice followed by trypsin inhibitor (100 μg/ml) to ensure detection of a protease-protected species. The percentages of individual proteins (KHNₜ CPY•, Ste6-166p, Bos1p, Erv25p, and Sec61 p) packaged into vesicles from a total reaction were determined by densitometric scanning of immunoblots. Protease protected [³⁵S]glyco-pro α-factor packaged into budded vesicles was measured by precipitation with concanavalin A-Sepharose after posttranslational translocation of [³⁵S)-prepro-α-F into microsomes (Wuestehube and Schekman, 1992). [³⁵S]glyco-pro-α factor was also visualized by Phosphorlmager analysis (Molecular Dynamics) after transfer to nitrocellulose membranes and exposure to a phosphor screen.

Indirect Immunofluorescence Microscopy

Cells were grown in synthetic complete medium to an OD₆₀₀ of 0.5-0.9. Formaldehyde (EM grade; Polysciences, Inc.) was added directly to the medium to 3.7% at 30° C. for 1 h. After fixation, cells were collected by centrifugation and washed with 5 ml 0.1 M potassium phosphate buffer (pH 7.5). Cells were incubated 30 min at 30° C. in spheroplasting buffer (1.0 mg/ml zymolyase 20T [ICN Biomedicals), 0.1 M potassium phosphate, pH 7.5, 0.1 % 2-mercaptoethanol) to digest the cell wall. Digestion was terminated by washing cells once in PBS. 30 μl of cell suspension was applied to each well of a poly-L-lysine-coated slide for 1 min and washed three times with PBS. Slides were immersed in acetone for 5 min at −20° C. and allowed to air dry. Subsequent steps were performed at room temperature. 30 −l of PBS block (3% BSA in PBS) were added to each well and incubated for 30 min. Primary antibodies α-HA or α-Kar2p were applied and used at 1:1,000 or 1:5,000 dilutions for in PBS block, respectively, for 1 h. Wells were washed three to five times with PBS block. 30 μl secondary antibodies (Alexa Fluor 488 goat α-mouse or α-rabbit and Alexa Fluor 546 goat α-mouse or α-rabbit; Molecular Probes, Inc.) were added to wells and incubated for 45 min in the dark. Wells were washed five to seven times with PBS block and two times with PBS. Each well is sealed with 5 μl mounting medium (PBS, 90°/glycerol, 0.025 μg/ml DAPI) and a glass coverslip. Samples were viewed on a ZEISS Axioplan epifluorescence microscope. Images were collected using a Spot 2 cooled digital camera (Diagnostic Instruments) and archived using Adobe Photoshopm 4.0. In experiments using KHNₜ, two copies of the gene were introduced into each strain to enhance detection. Low expression levels at single copy were likely due to suboptimal codon usage of this mammalian viral gene by yeast cells. By increasing gene dosage, the expression level was similar to CPY*_{HA} at single copy and had no effect on its processing as an ERAD substrate (unpublished data).

### REFERENCES

The following are hereby incorporated by reference in addition to all other referenced cited herein.

Barlowe, C., and R. Schekman. 1993. SEC12 encodes a guanine-nucleotide-exchange factor essential for transport vesicle budding from the ER Nature. 365:347-349.

Barlow, C., L. Ord, T. Young, M. Hosobuchi, S. Hamamoto, N. Salama, M. F. Rexach, M. Ravazzola, M. Amherdt, and R. Schekman. 1994. COPIL a membrane coat formed by Sec proteins that drive vesicle budding from the endoplasmic reticulum. Cell 77:895-907.

Bays, N. W., R. G. Gardner, L. P. Seelig, C. A. Joazeiro, and R. Y. Hampton. 2001. Hrdlp/Der3p is a membrane-anchored ubiquitin ligase required for ER-associated degradation. Nat. Cell Biol 3:24-29.

Belden, Wj., and C. Barlowe. 1996. Erv25p, a component of COPII-coated vesicles, forms a complex with Emp24p that is required for efficient endoplasmic reticulum to Golgi transport. J. Blot Qrm 271:26939-26946.

Biederer, T., C. Volkwein, and T. Sommer. 1996. Degradation of subunits of the Sec61p complex, an integral component of the ER membrane, by the ubiquitin-proteasome pathway. EMBO J. 15:2069-2076.

Biaderer, T., C. Volkwein, and T. Sommer. 1997. Role of Cue1 p in ubiquitination and degradation at the ER surface. Science. 27g:1go6-1809.

Bordallo, J., RK Plemper, A. Finger, and D. H. Wolf. 1998. Der3p/Hrd1p is required for endoplasmic reticulum-associated degradation of misfolded lumenal and integral membrane proteins. Mot Biol Cell 9:209-222.

Brodsky, J. L., and R Schekman. 1993. A Sec63p-BiP complex from yeast is required for protein translocation in a reconstituted proteoliposome. J. Cell Biot 123:1355-1363.

Brodsky, J. L., and A. A. McCracken. 1999. ER protein quality control and proteasome-mediated protein degradation. Semin. Cell Dev. Biol 10:507-513.

Brodsky, J. L., E. D. Werner, M. E. Dubas, J. L. Goeckeler, K. B. Kruse, and A. A. McCracken. 1999. The requirement for molecular chaperones during endoplasmic reticulum-associated protein degradation demonstrates that protein export and import are mechanistically distinct. J. Biol Chem. 274:34533460.

Caldwell, S. R, KJ. Hill, and A. A. Cooper. 2001. Degradation of ER quality control substrates require transport between the ER and Golgi. J. Biol Chem In press. 276:23296-23303.

Carrell, R. W., and B. Gooptu. 1998. Conformational changes and disease-serpins, prions and Alzheimer's. Cure Opin. Strucx Biol. 8:799-809.

Casagrande, R., P. Stem, M. Diehn, C. Sharon, M. Osario, M. Zuniga, P. O. Brown, and H. Ploegh. 2000. Degradation of proteins from the ER of S. cerevisiae requires an intact unfolded protein response pathway. Mol CeIL 5:729-735.

Cowles, C. R, W. B. Snyder, C. G. Burd, and S. D. Emr. 1997. Novel Golgi to vacuole delivery pathway in yeast: identification of a sorting determinant and required transport component. EMBO J. 16:2769-2782.

Eakle, K. A., M. Bernstein, and S. D. Emr. 1988. Characterization of a component of the yeast secretion machinery: identification of the SEC18 gene product. Mot Cell Not 8:4098-1109.

Ellgaard, L., M. Molinari, and A. Helenius. 1999. Setting the standards: quality control in the secretory pathway. Science. 286:1882-1888.

Elrod-Erickson, Mj., and C. A. Kaiser. 1996. Genes that control the fidelity of endoplasmic reticulum to Golgi transport identified as suppressors of vesicle budding mutations. Mol. Biol. Cell. 7:1043-1058.

Finger, A., M. Knop, and D. H. Wolf. 1993. Analysis of two mutated vacuolar proteins reveals a degradation pathway in the endoplasmic reticulum or a related compartment of yeast. Eur. J. Biochem. 218:565-574.

Friedlander, R, E. Jarosch, J. Urban, C. Volkwein, and T. Sommer. 2000. A regulatory link between ER-associated protein degradation and the unfolded protein response. Nat. Cell Not 2:379-384.

Gemmill, T. R, and RB. Trimble. 1999. Overview of N- and O-linked oligosaccharide structures found in various yeast species. Biochim. Biophys. Acta. 1426:227-237.

Gentzsch, M., and W. Tanner. 1996. The PMT gene family: protein O-glycosylation in Saccharomyces cerevisiae is vital. EMBO J. 15:5752-5759.

Gentzsch, M., T. Immervoll, and W. Tanner. 1995. Protein O-glycosylation in Saccharomyces cerevisiae: the protein O-mannosyltransferases Pmt1p and Pmt2p function as heterodimer. FEBS Lett. 377:128-130.

Gething, Mj., K. McCammon, and J. Sambrook. 1986. Expression of wild-type and mutant forms of influenza hemagglutinin: the role of folding in intracellular transport. Cell 46:939-950.

Hammond, C., and A. Helenius. 1994. Quality control in the secretory pathway: retention of a misfolded viral membrane glycoprotein involves cycling between the ER, intermediate compartment, and Golgi apparatus. J. Cell Blot 126:41-52.

Hampton, RY., R. G. Gardner, and J. Rine. 1996. Role of 26S proteasome and HRD genes in the degradation of 3-hydroxy-3-methylglutaryl-CoA reductase, an integral endoplasmic reticulum membrane protein. Mol. Biol. Cell 7:2029-2044.

Harty, C., S. Strahl, and K. Romisch. 2001. O-mannosylation protects mutant alpha-factor precursor from endoplasmic reticulum-associated degradation. Mol. Biol. Cell. 12:1093-1101.

Hauri, H., C. Appenzeller, F. Kuhn, and O. Nufer. 2000. Lectins and traffic in the secretory pathway. FEBS Lett. 476:32-37.

Herscovics, A., and P. Otlean. 1993. Glycoprotein biosynthesis in yeast. FASEB J 7:540-550.

Hiller, M. M., A. Finger, M. Schweiger, and D. H. Wolf. 1996. ER degradation of a mmisfolded luminal protein by the cytosolic ubiquitin-proteasome pathway. Science. 273:1725-1728.

Jakob, C. A., P. Burda, J. Roth, and M. Aebi. 1998. Degradation of misfolded endoplasmic reticulum glycoproteins in Saccharomyces cerevisiae is determined by a specific oligosaccharide structure. J. Cell Biol. 142:1223-1233.

Johnson, A. E., and M. A. van Waes. 1999. The translocon: a dynamic gateway at the ER membrane. Annu. Rev. Cell Dev. Biol. 15:799-442.

Johnson, A. E., and N. G. Haigh. 2000. The ER translocon and retrotranslocation: is the shift into reverse manual or automatic. Cell 102:709-712.

Kaiser, C. 2000. Thinking about p24 proteins and how transport vesicles select their cargo. Proc. Natl. Acad. Sci. USA. 97:3783-3785.

Katzmann, D. J., E. A. Epping, and W. S. Moye-Rowley. 1999. Mutational disruption of plasma membrane trafficking of Saccharomyces cerevisiae Yorlp, a homologue of mammalian multidrug resistance protein. Mol. Cell Biol. 19: 2998-3009.

Kim, P. S., and P. Arvan. 1998. Endocrinopathies in the family of endoplasmic reticulum (ER) storage diseases: disorders of protein trafficking and the role of ER molecular chaperones. Endocr. Rev. 19:173-202.

Knop, M., A. Finger, T. Braun, K. Hellmuth, and D. H. Wolf. 1996. Derl, a novel protein specifically required for endoplasmic reticulum degradation in yeast. EMBO J. 15:753 763.

Kopito, R. R., and D. Roes. 2000. Conformational disease. Nat. Cell Biol. 2:E207-E209.

Kozutsumi, Y., M. Segal, K. Normington, M. J. Gething, and J. Sambrook. 1988. The presence of malfolded proteins in the endoplasmic reticulum signals the induction of glucose-regulated proteins. Nature. 332:462-464.

Kreis, T. E., and H. F. Lodish. 1986. Oligomerization is essential for transport of vesicular stomatitis viral glycoprotein to the cell surface. Cell 46:929-937.

Lemurneur, F., E. C. Gaynor, S. Hennecke, C. Demolliere, R. Duden, S. D. Emr, H. Riezman, and P. Cosson. 1994. Coatomer is essential for retrieval of dilysine-tagged proteins to the endoplasmic reticulum. Cell 79:1199-1207.

Loayza, D., A. Tam, W. K. Schmidt, and S. Michaelis. 1998. Ste6p mutants defective in exit from the endoplasmic reticulum (ER) reveal aspects of an ER quality control pathway in Saccharomyces cerevisiae. Mol Biol. Cell. 9:2767-2784.

Lussier, M., A. M. Sdicu, F. Bmsereau, M. Jacquet, and H. Bussey. 1997. The Ktr1p, Ktr3p, and Kre2p/Mnt1p mannosyltransferases participate in the elaboration of yeast O- and N-linked carbohydrate chains. J. Biol Chem. 272:15527-15531.

Machamer, C. E., RW. Doms, D. G. Bole, A. Helenius, and J. K. Rose. 1990. Heavy chain binding protein recognizes incompletely disulfide-bonded forms of vesicular stomatitis virus G protein. J. Biol. Chem 265:6879-6883.

McCracken, A. A., and J. L. Brodsky. 1996. Assembly of ER-associated protein degradation in vitro: dependence on cytosol, calnexin, and ATP. J Cell Biol 132:291-298.

Muniz, M., C. Nuoffer, H. P. Hauri, and H. Riezman. 2000. The Emp24 complex recruits a specific cargo molecule into endoplasmic reticulum-derived vesicles. J. Cell Biol 148:925-930.

Muniz, M., P. Morsomme, and H. Riezman. 2001. Protein sorting upon exit from the endoplasmic reticulum. Cell 104:313-320.

Nakano, A., D. Brada, and R. Schekman. 1988. A membrane glycoprotein, Sec12p, required for protein transport from the endoplasmic reticulum to the Golgi apparatus in yeast. J. Cell Biol. 107:851-863.

Nehls, S., E. L. Snapp, N. B. Cole, K. J. Zaal, A. K. Kenworthy, T. H. Roberts, J. EIlenberg, J. F. Presley, E. Siggia, and J. Uppincott-Schwartz. 2000. Dynamics and retention of misfolded proteins in native ER membranes. Nat. Cell Biol. 2:288-295.

Ng, D. T. W., J. D. Brown, and P. Walter. 1996. Signal sequences specify the targeting route to the endoplasmic reticulum membrane. J. Cell Biol. 134:269278.

Ng, D. T. W., RE. Randall, and R. A. Lamb. 1989. Intracellular maturation and transport of the SV5 type II glycoprotein hemagglutinin-neuraminidase: specific and transient association with GRP78-BiP in the endoplasmic reticulum and extensive internalization from the cell surface. J Cell Biol. 109: 3273-3289.

Ng, D. T. W., S. W. Hiebert, and RA Lamb. 1990. Different roles of individual N-linked oligosaccharide chains in folding, assembly, and transport of the simian virus 5 hemagglutinin-neuraminidase. Mol. Cell Biol 10:198920o1.

Ng, D. T. W., E. D. Spear, and P. Walter. 2000. The unfolded protein response regulates multiple aspects of secretory and membrane protein biogenesis and endoplasmic reticulum quality control. J Cell Biol 150:77-88.

Nithikawa, S., A. Hirata, and A. Nakano. 1994. Inhibition of endoplasmic reticulum (ER)-to-Golgi transport induces relocalization of binding protein (BiP) within the ER to form the BiP bodies. Mol Biol. Cell 5:1129-1143.

Nithihawa, S., S. W. Fewdl, Y. Kato, J. L. Brodsky, and T. Endo. 2001. Molecular chaperones in the yeast endoplasmic reticulum maintain the solubility of proteins for retrotranslocation and degradation. J Cell Biol. 153:1-10.

Nuoffer, C., P. Jeno, A. Conzelmann, and H. Riezman. 1991. Determinants for glycophospholipid anchoring of the Saccharomyces cerevisiae GASI protein to the plasma membrane. Mol Cell Biol. 11:27-37.

Ogg, S. C., W. P. Barz, and P. Walter. 1998. A functional GTPase domain, but not its transmembrane domain, is required for function of the SRP receptor beta-subunit. J Cell Biol 142:341-354.

Parks, G. D., and RA. Lamb. 1990. Folding and oligomerization properties of a soluble and secreted form of the paramyxovirus hemagglutinin-neuraminidase glycoprotein. Virology. 178:498-508.

Pilon, M., R Schekman, and K. Romisch. 1997. Sec61p mediates export of a misfolded secretory protein from the endoplasmic reticulum to the cytosol for degradation. EMBOJ 16:4540-4548.

Plemper, RX, S. Bohntler, J. Bordallo, T. Sommer, and D. H. Wolf. 1997. Mutant analysis links the translocon and BiP to retrograde protein transport for ER degradation. Nature. 388:891-895.

Plemper, RK., P. M. Deak, RT. Otto, and D. H. Wolf. 1999. Re-entering the translocon from the lumenal side of the endoplasmic reticulum. Studies on mutated carboxypeptidase yscY species. FEBS Lett 443:241-245.

Sambrook, J., E. M. Fritsch, and T. Maniatis. 1989. Molecular Cloning: A Laboratory Manual. Cold Spring Harbor Laboratory Press, Plainview, N.Y.

Shimoni, Y., T. Kurihara, M. Ravazzola, M. Amherdt, L Ord, and R Schekman. 2000. Lstip and Sec24p cooperate in sorting of the plasma membrane ATPase into COPII vesicles in Saccharomyces cerevisiae. J Cell Biol 151: 973-984.

Sikorski, RS., and P. Hieter. 1989. A system of shuttle vectors and yeast host strains designed for efficient manipulation of DNA in Saccharomyces cerevisiae. Genetics. 122:19-27.

Sommer, T., and S. Jentsch. 1993. A protein translocation defect linked to ubiquitin conjugation at the endoplasmic reticulum. Nature. 365:176-179.

Sommer, T., and D. H. Wolf. 1997. Endoplasmic reticulum degradation: reverse protein flow of no return. FASEB J. 11:1227-1233.

Spormann, D. O., J. Heim, and D. H. Wolf. 1992. Biogenesis of the yeast vacuole (lysosome). The precursor forms of the soluble hydrolase carboxypeptidase yscS are associated with the vacuolar membrane. J. Biol Chem. 267:80218029.

Strahl-Bolsinger, S., M. Gentasch, and W. Tanner. 1999. Protein O-mannosylation. Biochim Biophys Acta. 1426:297-307.

Travers, K. J., C. K. Patil, L. Wodicka, D. J. Lockhart, J. S. Weissman, and P. Walter. 2000. Functional and genomic analyses reveal an essential coordination between the unfolded protein response and ER-associated degradation. Cell 101:249-258.

Wang, Q, and A. Chang. 1999. Epsl, a novel PDI-related protein involved in ER quality control in yeast. EMBO J. 18:5972-5982.

Ward, C. L., S. Omura, and RR Kopito. 1995. Degradation of CFTR by the ubiquitin-proteasome pathway. Cell 83:121-127.

Werner, E. D., J. L. Brodsky, and A. A. McCracken. 1996. Proteasome-dependent endoplasmic reticulum-associated protein degradation: an unconventional route to a familiar fate. Proc. Natl Acad Sci. USA. 93:13797-13801.

Wiertz, E. J., D. Tortorella, M. Bogyo, J. Yu, W. Mothes, T. R Jones, T. A. Rapoport, and H. L. Ploegh. 1996. Sec61-mediated transfer of a membrane protein from the endoplasmic reticulum to the proteasome for destruction. Nature. 384:432-438.

Wuestehube, L. J., and RW. Schekman. 1992. Reconstitution of transport from endoplasmic reticulum to Golgi complex using endoplasmic reticulumenriched membrane fraction from yeast. Methods Enzymol 219:124-136.

Zhou, M., and R Schekman. 1999. The engagement of Sec61p in the ER dislocation process. Mol Cell 4:925-934.

### Example 2

A Yeast Vector System for the Expression of Eukaryotic Secretory Proteins.

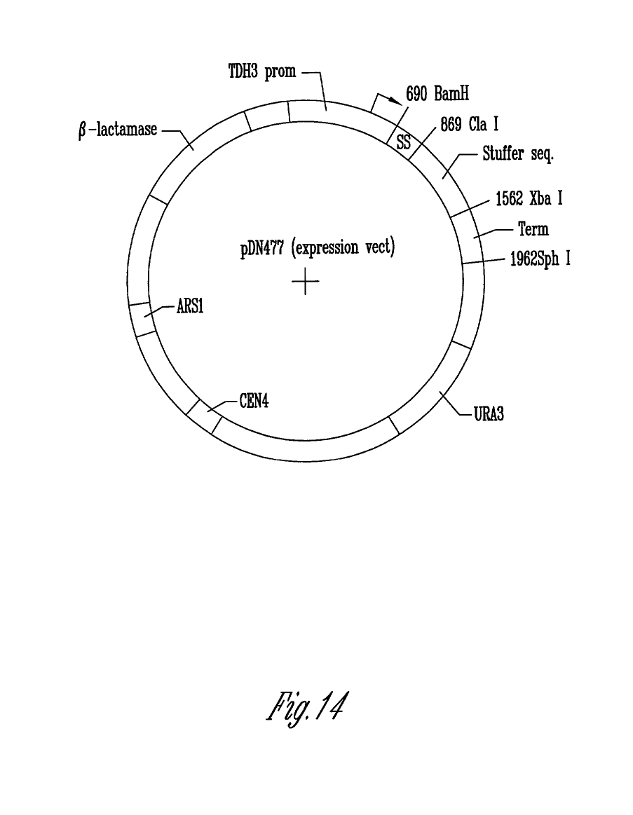

We have designed and constructed a versatile vector system See FIG. 14 for the expression of heterologous (e.g., mammalian) secreted and membrane proteins in yeast. The vector contains a bacterial replicon for propagation and manipulation in E. coli. It also contains a yeast origin and centromere for replication and mitotic stability. Alternatively, a version is available for genomic integration to generate stable strains. The expression module can be easily manipulated depending on the needs of the user. Expression is driven from the TDH3 promoter, the strongest known constitutive promoter in S. cerevisiae. Strategically placed restriction sites allow the use of the subject's own signal sequence (if determined to be functional in yeast) or the yeast BiP signal sequence contained in the module. The yeast BiP signal sequence has proven to be more effective than others since it directs the recombinant protein into the SRP pathway, a cotranslational translocation mechanism that is the primary pathway used by secreted and membrane proteins in mammalian cells. The commonly used alpha-Factor signal sequence has proven to be problematic since it uses a posttranslational pathway that is uncommon in higher eukaryotes. By contrast, a 100% success rate in the efficacy of the BiP signal sequence was shown for expressing heterologous proteins. The module also contains a 6-histidine tag to facilitate purification of the recombinant protein. The tag can be removed during insertion of the subject cDNA if not required. Transcription is terminated by the yeast ACT1 terminator.

What is claimed is:

1. A method of producing a heterologous protein in fungi comprising:
providing a recipient fungi cell wherein the quality control mechanism in said cell is modified so that incompletely folded heterologous proteins are not degraded in the endoplasmic reticulum; and
introducing to said recipient fungi cell a polynucleotide expression construct.

2. The method of claim 1 wherein said fungi cell is a yeast cell.

3. The method of claim 1 wherein said introducing is by a transformation method selected from the group consisting of:
PEG, electroporation, particle bombardment, and LiAc.

4. The method of claim 3 wherein said transformation method is LiAc mediated transformation.

5. The method of claim 1 wherein said polynucleotide construct is within a yeast based plasmid.

6. The method of claim 1 wherein said recipient cell is modified so that O-glycosylation is inhibited.

7. The method of claim 6 wherein said recipient cell comprises inhibition of a protein mannosyltransferase gene.

8. The method of claim 7 wherein said mannosyltransferase gene comprises a gene selected from the group consisting of PMT 1, PMT 2, PMT 3, PMT 4, PMT 5, AND PMT 6.

9. The method of claim 8 wherein said PMT gene is PMT 1.

10. The method of claim 8 wherein said PMT gene is PMT 2.

11. The method of claim 8 wherein said recipient gene provides inhibition of a Bypass of Sec Thirteen gene.

12. The method of claim 11 wherein said Bypass of Sec Thirteen gene is BST1.

13. A yeast cell transformed by the method of claim 1.

14. A protein produced by the method of claim 1.

15. A method of producing a heterologous protein in a fungi cell comprising:
providing a recipient fungi cell wherein o-glycosylation is inhibited so that misfolded heterologous proteins are not degraded; and
introducing to said recipient fungi cell a polynucleotide expression construct, said construct comprising a structural gene to be expressed in said cell, said gene operably linked to control sequences for expression in a fungi cell wherein said recipient fungi cell.

16. The method of claim 15 wherein said fungi cell is a yeast cell.

17. The method of claim 15 wherein said introducing is by a transformation method selected from the group consisting of:
PEG, electroporation, particle bombardment, and LiAc.

18. The method of claim 17 wherein said transformation method is LiAc mediated transformation.

19. The method of claim 15 wherein said polynucleotide construct is within a yeast based plasmid.

20. The method of claim 15 wherein said recipient cell comprises a protein mannosyltransferase gene the expression of which is inhibited.

21. The method of claim 20 wherein said mannosyltransferase gene comprises a gene selected from the group consisting of PMT 1, PMT 2, PMT 3, PMT 4, PMT 5, AND PMT 6.

22. The method of claim 15 wherein said PMT gene is PMT 1.

23. The method of claim 15 wherein said PMT gene is PMT 2.

24. A yeast cell transformed by the method of claim 15.

25. A protein produced by the method of claim 15.

26. A method of producing a heterologous protein in fungi comprising:
providing a recipient fungi cell wherein Bypass of Sec Thirteen expression is inhibited so that misfolded heterologous proteins are not degraded; and
introducing to said recipient fungi cell a polynucleotide expression construct, said construct comprising a structural gene to be expressed in said cell, said gene operably linked to control sequences for expression in a fungi cell wherein said recipient fungi cell.

27. The method of claim 1 wherein said fungi cell is a yeast cell.

28. The method of claim 1 wherein said introducing is by a transformation method selected from the group consisting of: PEG, electroporation, particle bombardment, and LiAc.

29. The method of claim 28 wherein said transformation method is LiAc mediated transformation.

30. The method of claim 28 wherein said polynucleotide construct is within a yeast based plasmid.

31. The method of claim 28 wherein said Bypass of Sec Thirteen gene is BST1.

32. A yeast cell transformed by the method of claim 28.

33. A protein produced by the method of claim 28.

34. A polynucleotide useful for transforming yeast cells comprising:
A promoter capable of driving expression in a yeast cell
A bacterial replicon for propagation in E. Coli,
A transcription termination signal;
A yeast BiP signal sequence;
A yeast origin and centromere for replication and mitotic stability, wherein said polynucleotide directs expression of the recombinant protein to the SRP pathway.

35. The polynucleotide of claim 34 further comprising a 6histidine tag to facilitate protein purification.

36. The polynucleotide of claim 34 wherein the vector is as depicted in FIG. 14.

37. A yeast cell for production of heterologous proteins, said cell comprising a modification so that a quality control mechanism in said cell is modified so that misfolded heterologous proteins are not degraded in the endoplasmic reticulum.

38. The yeast cell of claim 37 wherein said modification comprises a modification that inhibits of O-linked glycosylation.

39. The yeast cell of claim 38 wherein said modification is a PMT loss of function modification.

40. The yeast cell of claim 39 wherein said PMT modification is to PMT 1.

41. The yeast cell of claim 41 wherein said PMT modification is to PMT 2

42. The yeast strain of claim 37 wherein said modification comprises a modification that inhibits the production of Bypass of Sec Thirteen.
